(19) United States
(12) Patent Application Publication
(10) Pub. No.: US 2024/0325294 A1
(43) Pub. Date: Oct. 3, 2024

(54) ETONOGESTREL FORMULATION

(71) Applicant: MEDINCELL SA, Jacou (FR)

(72) Inventors: Maria FERRAND, Jacou (FR); Sophie LELAMER, Jacou (FR); Elodie REY, Jacou (FR); Lilian SANTA, Jacou (FR); Myriam ABBASSI, Jacou (FR)

(21) Appl. No.: 18/557,882

(22) PCT Filed: Apr. 29, 2022
(86) PCT No.: PCT/EP2022/061511

§ 371 Date: Oct. 27, 2023

(30) Foreign Application Priority Data
Apr. 30, 2021 (EP) 21305559.3

(51) Int. Cl. A61K 9/00; A61K 31/567; A61K 47/10; A61K 47/34

(52) U.S. Cl. CPC A61K 9/0024; A61K 31/567; A61K 47/10; A61K 47/34

(57) ABSTRACT

The present invention generally relates to an injectable formulation for sustained release of etonogestrel, a pharmaceutical dosage and an application device comprising such formulation, methods for preparing thereof, and use thereof for contraception.

### FIELD OF THE INVENTION

The present invention generally relates to an injectable formulation for sustained release of etonogestrel, a pharmaceutical dosage and an application device comprising such formulation, methods for preparing thereof, and use thereof for contraception.

### BACKGROUND ART

To date, approximately 214 million women in developing countries have an unmet need for modern contraception. This is the result of the combination between the lack of access to contraceptive supplies, and deficiency of family planning education and information regarding how contraceptive methods work. As a result, enabling women to act on their pregnancy choice has become a high priority on the global development agenda. There is a particular demand for development of new contraceptive technologies, including long-acting injectable (LAI) methods.

LAIs are among the most convenient, effective and safe contraception products, which gives them justifiably wide appeal amongst many groups of women. Currently, existing birth control implants are capable of providing a secure contraception through a 3-5 years of sustained release of progestins. For example, Nexplanon® is a progestin-only implant for subdermal use. The implant is a non-biodegradable rod, 4 cm in length with a diameter of 2 mm, and consists of an ethylene vinyl acetate (EVA) copolymer core, containing etonogestrel, barium sulfate (radiopaque ingredient), and may also contain magnesium stearate, surrounded by an EVA copolymer skin.

Though, the long duration of contraception action, in many cases fails to meet the needs and preferences of women desiring access to family planning tools and interventions. Solid rod implants may require surgical implantation, and can cause inconvenience or break within the body of the patient. In addition, rod implants are costly and not biodegradable. Consequently, implant removal surgery (explantation) by a specially trained doctor or nurse is necessary.

As such, there is still an unmet need for a contraceptive providing secure contraception over a moderately long duration (multiple months), the handling of which is less burdensome, to enable access of women to family planning methods with more flexibility, address barriers to nonuse, and accelerate the acceptance for and uptake of contraceptives for women. That is, the contraceptive should provide, on the one hand, sufficient contraceptive effect over multiple months, but, on the other hand, over a shorter overall period as compared to existing implants, to offer the flexibility for women to stop contraception if they want to conceive (i.e., planning flexibility) moderately rapidly and easily (i.e., without need for surgical implant removal). Yet further, there is a demand for a contraceptive sufficiently stable to be transported or stored at elevated temperatures, given that need for contraception is unmet particularly in multiple developing countries, many of which with a warm climate. And, there is a demand for a contraceptive obtainable by robust, scalable manufacturing methods.

### Problem Underlying the Invention

In view of the above background, the present invention aims to provide an improved contraceptive formulation or pharmaceutical dosage form acting over multiple months, the handling of which is less burdensome; an application device allowing easy administration of the formulation or dosage form to a subject, a robust and scalable method for producing the formulation, and use thereof as contraception allowing better planning flexibility.

### SUMMARY OF THE INVENTION

As a solution, the present invention provides an injectable formulation for sustained release of etonogestrel, comprising:
a triblock copolymer (TB) having the formula A-B-A′, wherein:
blocks A and A′ are formed of poly(D,L-lactic acid), and comprise, respectively, m and n lactic acid (LA) repeating units;
block B is formed of poly(ethylene glycol) comprising p ethylene oxide (EO) repeating units; and
the molar ratio R(TB) of LA to EO repeating units ((m+n)/p) is 1.5 to 9;
a diblock copolymer (DB) having the formula C-D, wherein:
block C is formed of methoxypoly(ethylene glycol) comprising r EO repeating units;
block D is formed of poly(D,L-lactic acid) comprising q LA repeating units; and
the molar ratio R(DB) of LA to EO repeating units (q/r) is 1.5 to 15;
a water-miscible, pharmaceutically acceptable organic solvent comprising DMSO; and
etonogestrel, prodrug, or salt thereof.

The present invention further provides a pharmaceutical dosage form comprising the above formulation.

The present invention further provides a method for preparing the above formulation and/or dosage form, comprising the steps of:
(i) mixing:
   a triblock copolymer (TB) having the formula A-B-A′, wherein blocks A and A′ are formed of poly(D,L-lactic acid) and comprise, respectively, m and n lactic acid (LA) repeating units; block B is formed of poly(ethylene glycol) comprising p ethylene oxide (EO) repeating units; the molar ratio R(TB) of LA to EO repeating units (m+n)/p is 1.5 to 9;
   a diblock copolymer (DB) having the formula C-D, wherein block C is formed of methoxypoly(ethylene glycol) comprising r EO repeating units; block D is formed of poly(D,L-lactic acid) comprising q LA repeating units; and the molar ratio R(DB) of LA to EO repeating units (q/r) is 1.5 to 12; and
   a water-miscible, pharmaceutically acceptable organic solvent comprising DMSO, until the polymers are dissolved, thereby obtaining a vehicle; and
(ii) mixing the vehicle with etonogestrel, thereby obtaining the formulation;
(iii) optionally sterilizing the formulation; and
(iv) providing, and optionally packaging, a predetermined amount of the formulation, thereby obtaining the dosage form.

The present invention further provides an application device comprising the above formulation and/or dosage form, the device being adapted for administering the formulation or dosage form to a subject by injection. The present invention further provides a use of the above formulation, dosage form or device for contraception in a female subject.

### BRIEF DESCRIPTION OF THE DRAWINGS

FIG. 1A shows the in vitro release rates of etonogestrel over time obtained from different formulations at a 10 mg dose. Formulations are composed of 1.5 to 10% w/w of etonogestrel, 30 to 50% w/w of polymers, and 45 to 60% DMSO. Different diblocks and triblocks were used. Formulation compositions are detailed in Table 1. In vitro release tests were performed as explained in Example 2.

FIG. 1B shows the in vitro cumulative etonogestrel release profiles obtained from different formulations at a 10 mg dose. Formulations are composed of 1.5 to 10% w/w of etonogestrel, 30 to 50% w/w of polymers, and 45 to 60% DMSO. Different diblocks and triblocks were used. Formulation compositions are detailed in Table 1. In vitro release tests were performed as explained in Example 2.

FIG. 2A shows the in vitro release rates of etonogestrel over time obtained from different formulations at a 15 mg dose. Formulations are composed of 7.5 to 10% w/w of etonogestrel, 34 to 42% w/w of polymers, and 50 to 56% DMSO. Diblock and triblock were fixed as well as their relative ratio. Formulation compositions are detailed in Table 1. In vitro release tests were performed as explained in Example 2.

FIG. 2B shows the in vitro cumulative etonogestrel release profiles obtained from different formulations at a 15 mg dose. Formulations are composed of 7.5 to 10% w/w of etonogestrel, 34 to 42% w/w of polymers and 50 to 56% DMSO. Diblock and triblock were fixed as well as their relative ratio. Formulation compositions are detailed in Table 1. In vitro release tests were performed as explained in Example 2.

FIG. 3A shows the in vitro release rates of etonogestrel over time obtained from the same formulation at different doses varying from 10 to 40 mg. Formulation composition is detailed in Table 1. in vitro release tests were performed as explained in Example 2.

FIG. 3B shows the in vitro cumulative etonogestrel release profiles obtained from the same formulation at different doses varying from 10 to 40 mg. Formulation composition is detailed in Table 1. In vitro release tests were performed as explained in Example 2.

FIG. 4 presents the mean kinetic profiles of etonogestrel obtained after a single subcutaneous injection of formulations #3, #7 or #9. Different sustained release profiles were obtained with the 3 test items, with a quantifiable level of etonogestrel for up to 180 days with the tested 15 mg dose. Formulation compositions are detailed in Table 1. Pharmacokinetic studies were performed as explained in Example 3.

FIG. 5 presents the mean kinetic profiles of etonogestrel obtained after a single subcutaneous injection of formulations #15, #16, #17 or #18. Different sustained release profiles were obtained with the 4 test items, with a quantifiable level of etonogestrel for up to 224 days with #18. Formulation compositions are detailed in Table 1, diblock and triblock were fixed as well as their relative ratio. Pharmacokinetic studies were performed as explained in Example 3.

### DETAILED DESCRIPTION OF THE INVENTION

The contraceptive formulation, dosage form, method for preparation, application device and use according to the present invention provide multiple advantageous effects.

Owing to the specific combination of its ingredients, the formulation is capable of releasing etonogestrel in sustained and controlled manner and providing contraceptive effect, characterized by low initial burst, and/or extended, controlled release of etonogestrel over multiple months (e.g., at least 120 days).

The formulation of the present invention is an injectable liquid. That is, the formulation has a sufficiently low viscosity allowing for easy administration by injection. As such, administration can be performed even without the necessity of involving a doctor or nurse. The handling and application are thus less burdensome. Accordingly, the application device adapted for injection allows easy administration of the formulation or dosage form to a subject.

The formulation of the present invention forms, upon injection, a depot in situ, and delivers etonogestrel to the subject.

The formulation of the present invention is biodegradable. As opposed to solid, non-biodegradable implants, this eliminates the necessity for implant removal surgery (explantation) by a specially trained doctor, nurse, or healthcare professional, and further contributes to improved ease of application for the subject.

The formulation is a liquid. It is capable of being filtered, for instance, by means of sterile filtration. This enables robust and scalable purification during production, and in particular, sterilization by means of filtration. The formulation exhibits good filtration stability.

The formulation also exhibits good storage stability. For instance, the formulation may exhibit good stability under long-term stability condition in an oven set at 30° C.±3° C., and/or accelerated stability condition in ICH climatic chambers (40° C.±2° C./75% RH±5% RH), as confirmed, e.g., by visual appearance, active ingredient assay, and related substances (UV-UPLC), water content (Karl Fischer volumetric titration), injectability (syringe injection force), copolymer integrity (dynamic viscosity, and molecular weight by GPC-RI), or in vitro release (e.g., repeatability over at least 30 days).

The formulation may also be compatible with dry heat sterilization. For instance, the formulation May exhibit good stability as evaluated through the stability assessment after dry heat sterilization cycles (e.g., +121° C./35 min, and +111° C./60 min).

Specifically, the present invention provides the following aspects and embodiments.
1. Injectable formulation for sustained release of etonogestrel, comprising:
   a triblock copolymer (TB) having the formula A-B-A′, wherein:
      blocks A and A′ are formed of poly(D,L-lactic acid), and comprise, respectively, m and n lactic acid (LA) repeating units;
      block B is formed of poly(ethylene glycol) comprising p ethylene oxide (EO) repeating units; and
      the molar ratio R(TB) of LA to EO repeating units ((m+n)/p) is 1.5 to 9;
   a diblock copolymer (DB) having the formula C-D, wherein:
      block C is formed of methoxypoly(ethylene glycol) comprising r EO repeating units;
      block D is formed of poly(D,L-lactic acid) comprising q LA repeating units; and the molar ratio R(DB) of LA to EO repeating units (q/r) is 1.5 to 15;
   a water-miscible, pharmaceutically acceptable organic solvent comprising DMSO; and
   etonogestrel, prodrug, or salt thereof.
2. The formulation according to aspect 1, wherein block B has a number-average molecular weight (Mₙ(B)) of 500 to 3,500 g/mol; and/or block C has a number-average molecular weight (Mₙ(C)) of 175 to 3,000 g/mol.
3. The formulation according to aspect 1 or 2, wherein:
   the triblock copolymer (TB) has a number-average molecular weight (Mₙ(TB)) of 1,700 to 48,000 g/mol, preferably 1,800 to 45,000 g/mol, more preferably 4,500 to 40,000 g/mol, more preferably 6,000 to 30,000 g/mol, more preferably 7,000 to 20,000 g/mol, more preferably 9,000 to 18,000 g/mol, more preferably 14,000 to 16,000 g/mol, most preferably 11,000 to 15,000 g/mol; and/or
   the diblock copolymer (DB) has a number-average molecular weight (Mₙ(DB)) of 650 to 77,000 g/mol, preferably 1,000 to 50,000 g/mol, more preferably 2,000 to 30,000 g/mol, more preferably 3,000 to 15,000 g/mol, more preferably 4,000 to 11,000 g/mol, more preferably 6,000 to 12,000, more preferably 5,000 to 8,000 g/mol, more preferably 6,000 to 8,000 g/mol, or most preferably 5,500 to 7,500 g/mol.
4. The formulation according to any one of the preceding aspects, wherein:
   the triblock copolymer (TB) has a weight-average molecular weight (M_{w}(TB)) of 1,700 to 96,000 g/mol, preferably 1,800 to 90,000 g/mol, more preferably 4,500 to 80,000 g/mol, more preferably 6,000 to 60,000 g/mol, more preferably 7,000 to 40,000 g/mol, more preferably 9,000 to 36,000 g/mol, more preferably 14,000 to 32,000 g/mol; most preferably 11,000 to 30,000 g/mol; and/or
   the diblock copolymer (DB) has a weight-average molecular weight (M_{w}(DB)) of 650 to 154,000 g/mol, preferably 1,000 to 100,000 g/mol, more preferably 2,000 to 60,000 g/mol, more preferably 3,000 to 30,000 g/mol, more preferably 4,000 to 22,000 g/mol, more preferably 6,000 to 24,000, more preferably 5,000 to 16,000 g/mol, more preferably 6,000 to 16,000, or most preferably 5,500 to 15,000 g/mol.
5. The formulation according to any one of the preceding aspects, wherein Mₙ(B) is 500 to 3,500, preferably 650 to 3,000, more preferably 650 to 2,500, more preferably 800 to 2,200, more preferably 800 to 2,000, more preferably 950 to 1050, more preferably 1,000 to 2,000, most preferably 1,000 to 1,500 or 1,500 to 2,000 g/mol; and/or R(TB) is 1.5 to 8.0, preferably 2.5 to 8.0, more preferably 2.5 to 7.5, more preferably 3.5 to 7.5, more preferably 3.5 to 7.0, most preferably 4.5 to 7.5.
6. The formulation according to any one of the preceding aspects, wherein Mₙ(C) is 175 to 3,000, preferably 200 to 3,000, more preferably 200 to 2,500, more preferably 250 to 2,500, more preferably 250 to 2,500, more preferably 350 to 2,000, more preferably 330 to 370, most preferably 350 to 1,200 or 1,200 to 2,000 g/mol; and/or R(DB) is 1.8 to 12.0, preferably 1.8 to 11.0, more preferably 2.5 to 11.0, more preferably 2.5 to 10.0, more preferably 3.0 to 10.0, more preferably 3.0 to 9.5, most preferably 6.5 to 10.5.
7. The formulation according to any one of the preceding aspects, wherein:
(a) Mₙ(B) is 500 to 2,000, preferably 600 to 1,800, more preferably 650 to 1,500, most preferably 800 to 1,200 g/mol; R(TB) is 1.5 to 6, preferably 2.5 to 6, more preferably 2.5 to 5, most preferably 3.5 to 4.5; Mₙ(C) is 700 to 3,000, preferably 700 to 2,500, more preferably 1,500 to 2,500, most preferably 1,800 to 2,200 g/mol; and R(DB) is 2 to 12, preferably 2.5 to 5, more preferably 2.5 to 3.5, most preferably 2.7 to 3.3; or
(b) Mₙ(B) is 500 to 2,000, preferably 600 to 1,800, more preferably 650 to 1,500, most preferably 800 to 1,200 g/mol; R(TB) is 1.5 to 8, preferably 4 to 8, more preferably 4.5 to 7.5, most preferably 5.5 to 6.5; Mₙ(C) is 200 to 3,000, preferably 200 to 1,500, more preferably 250 to 1,500, most preferably 250 to 500 g/mol; and R(DB) is 2.5 to 12, preferably 2.5 to 11, more preferably 6.5 to 10.5, most preferably 7.5 to 9.5; or
(c) Mₙ(B) is 1,200 to 3,200, preferably 1,200 to 2,500, more preferably 1,500 to 2,500, most preferably 1,800 to 2,200 g/mol; R(TB) is 1.5 to 8, preferably 5 to 8, more preferably 4 to 8, most preferably 3.2 to 3.8; Mₙ(C) is 175 to 2,500, preferably 250 to 2,500, more preferably 250 to 2,400, most preferably 300 to 2,400 g/mol; and R(DB) is 2 to 12, preferably 2.5 to 10, more preferably 2.5 to 10, most preferably 2.7 to 9.
8. The formulation according to any one of the preceding aspects, wherein Mₙ(B) is 500 to 1,800, preferably 650 to 1,800, more preferably 650 to 1,500, most preferably 800 to 1,200 g/mol.
9. The formulation according to any one of the preceding aspects, wherein Mₙ(C) is 200 to 1,500, preferably 200 to 500, most preferably 250 to 500 g/mol.
10. The formulation according to any one of the preceding aspects, wherein the etonogestrel content is 2.5% w/w or more, or 15% w/w or less, preferably 2.5 to 15% w/w, more preferably 4 to 15% w/w, more preferably 4 to 12% w/w, more preferably 5 to 12% w/w, more preferably 5 to 10% w/w, more preferably 6 to 10% w/w, more preferably 6 to 10% w/w, most preferably 7 to 10% w/w, relative to the total weight of the formulation.
11. The formulation according to any one of the preceding aspects, wherein the triblock copolymer (TB) content is 8% w/w or more, or 40% w/w or less, preferably 8 to 40% w/w, more preferably 9 to 40% w/w, more preferably 9 to 35% w/w, more preferably 10 to 35% w/w, more preferably 10 to 30% w/w, more preferably 15 to 30% w/w, more preferably 15 to 25% w/w, most preferably 17 to 21% w/w, relative to the total weight of the formulation.
12. The formulation according to any one of the preceding aspects, wherein the diblock copolymer (DB) content is 6% w/w or more, or 50% w/w or less, preferably 6 to 50% w/w, preferably 10 to 50% w/w, more preferably 10 to 45% w/w, more preferably 15 to 40% w/w, more preferably 15 to 40% w/w, more preferably 17 to 40% w/w, more preferably 17 to 30% w/w, most preferably 17 to 21% w/w, relative to the total weight of the formulation.
13. The formulation according to any one of the preceding aspects, wherein the total content of polymers TB and DB in the formulation is 14% w/w or more or 55% w/w or less, more preferably 18 to 55% w/w, more preferably 18 to 50% w/w, more preferably 30 to 55% w/w, more preferably 30 to 50% w/w, more preferably 32 to 50% w/w, more preferably 32 to 45% w/w, most preferably 34 to 42% w/w, relative to the total weight of the formulation.
14. The formulation according to any one of the preceding aspects, wherein the weight ratio of triblock to diblock copolymer (TB:DB) is 10:90 w/w or more, or 70:30 w/w or less, preferably 10:90 to 70:30 w/w, more preferably 10:90 to 65:35 w/w, more preferably 15:85 to 65:35 w/w, more preferably 15:85 to 60:40 w/w, more preferably 20:80 to 60:40 w/w, more preferably 20:80 to 55:45 w/w, more preferably 42:58 to 58:42 w/w, most preferably 47:53 to 53:47 w/w.
15. The formulation according to any one of the preceding aspects, wherein the water-miscible, pharmaceutically acceptable organic solvent content is 40% w/w or more, or 82.5% w/w or less, preferably 45 to 82.5% w/w, more preferably 45 to 78% w/w, more preferably 40 to 78% w/w, more preferably 40 to 65% w/w, more preferably 45 to 65% w/w, more preferably 40 to 59% w/w, more preferably 46 to 57% w/w, most preferably 50 to 56% w/w, relative to the total weight of the formulation.
16. The formulation according to any one of the preceding aspects, wherein the water-miscible, pharmaceutically acceptable organic solvent essentially consists of DMSO, or is a mixture of DMSO and one or more co-solvents, preferably N-methyl pyrrolidone (NMP); more preferably wherein the water-miscible, pharmaceutically acceptable organic solvent is DMSO.
17. The formulation according to any one of the preceding aspects, further comprising one or more additional pharmaceutically acceptable excipient(s), such as polylactic acid (PLA), polyethylene glycol or poly(lactic-co-glycolic acid) (PLGA).
18. The formulation according to any one of the preceding aspects, wherein: Mₙ(B) is 500 to 1,800, preferably 650 to 1,800, more preferably 650 to 1,500, most preferably 800 to 1,200 g/mol; R(TB) is 1.5 to 6, preferably 2.5 to 6, more preferably 2.5 to 5, most preferably 3.5 to 4.5; Mₙ(C) is 700 to 3,000, preferably 700 to 2,500, more preferably 1,500 to 2,500, most preferably 1,800 to 2,200 g/mol; and R(DB) is 2 to 12, preferably 2.5 to 5, more preferably 2.5 to 3.3, most preferably 2.7 to 3.3; and the etonogestrel content is 7% w/w or more, preferably 7.5% w/w or more, more preferably 8% w/w or more, more preferably 9% w/w or more, more preferably 10% w/w or more, relative to the total weight of the formulation.
19. The formulation according to any one of the preceding aspects, wherein: Mₙ(B) is 500 to 1,800, preferably 650 to 1,800, more preferably 650 to 1,500, most preferably 800 to 1,200 g/mol; R(TB) is 1.5 to 8, preferably 2.5 to 8, more preferably 3.5 to 7.5, most preferably 4.5 to 7.5; Mₙ(C) is 175 to 3,000, preferably 200 to 1,500, more preferably 200 to 500, most preferably 250 to 500 g/mol; and R(DB) is 2.0 to 12.0, preferably 3.0 to 10.0, more preferably 3.0 to 9.5, most preferably 6.5 to 10.5; and the etonogestrel content is 7% w/w or more, preferably 7.5% w/w or more, more preferably 8% w/w or more, more preferably 9% w/w or more, more preferably 10% w/w or more, relative to the total weight of the formulation.
20. The formulation according to any one of the preceding aspects, wherein: Mₙ(B) is 500 to 1,800, preferably 650 to 1,800, more preferably 650 to 1,500, most preferably 800 to 1,200 g/mol; R(TB) is 1.5 to 8, preferably 2.5 to 8, more preferably 3.5 to 7.5, most preferably 4.5 to 7.5; Mₙ(C) is 175 to 3,000, preferably 200 to 1,500, more preferably 200 to 500, most preferably 250 to 500 g/mol; and R(DB) is 2.0 to 12.0, preferably 3.0 to 10.0, more preferably 3.0 to 9.5, most preferably 6.5 to 10.5; and the etonogestrel content is 7% w/w or more and 10% w/w or less, preferably 7% w/w or more and 9% w/w or less, most preferably 7.5% w/w or more and 8% w/w or less, relative to the total weight of the formulation.
21. The formulation according to any one of the preceding aspects, wherein the etonogestrel content is 7% w/w or more, preferably 7.5% w/w or more, preferably 8% w/w or more, more preferably 8% w/w or more, more preferably 9% w/w or more, more preferably 10% w/w or more, relative to the total weight of the formulation; and the water-miscible, pharmaceutically acceptable organic solvent content is 56% w/w or less, preferably 55% w/w or less, more preferably 54% w/w or less, relative to the total weight of the formulation.
22. The formulation according to any one of the preceding aspects, wherein the etonogestrel content is 7% w/w or more, preferably 7.5% w/w or more, relative to the total weight of the formulation; and the water-miscible, pharmaceutically acceptable organic solvent content is 56% w/w or less, preferably 54% w/w or less, relative to the total weight of the formulation.
23. The formulation according to any one of the preceding aspects, wherein the triblock copolymer (TB) content is 15% w/w or more, preferably 16% w/w or more, more preferably 17% w/w or more, relative to the total weight of the formulation; and the diblock copolymer (DB) content is 35% w/w or less, preferably 30% w/w or less, more preferably 25% w/w or less, relative to the total weight of the formulation; and the weight ratio of triblock to diblock copolymer (TB:DB) is 30:70 w/w or higher, preferably 40:60 w/w or higher, more preferably 50:50 w/w or higher.
24. The formulation according to any one of the preceding aspects, wherein the total content of polymers TB and DB in the formulation is 30% w/w or more, preferably 32% w/w or more, more preferably 34% w/w or more, relative to the total weight of the formulation.
25. The formulation according to aspect 23 or 24, wherein the triblock copolymer (TB) content is 40% w/w or less, preferably 35% w/w or less, more preferably 30% w/w or less, relative to the total weight of the formulation; and the diblock copolymer (DB) content is 17% w/w or more, preferably 18.5% w/w or more, more preferably 20% w/w or more and 50% w/w or less, relative to the total weight of the formulation.
26. The formulation according to any one of aspects 23 to 25, wherein the total content of polymers TB and DB in the formulation is 45% w/w or less, preferably 43% w/w or less, more preferably 40% w/w or less, and 35% w/w or more, preferably 38% w/w or more, more preferably 40% w/w or more, relative to the total weight of the formulation; and the weight ratio of triblock to diblock copolymer (TB:DB) is 45:55 w/w or higher, preferably 47:53 w/w or higher, more preferably 50:50 w/w or higher.
27. The formulation according to any one of the preceding aspects, wherein the total content of polymers TB and DB in the formulation is 48% w/w or less, preferably 45% w/w or less, preferably 43% w/w or less, more preferably 40% w/w or less, relative to the total weight of the formulation; and the weight ratio of triblock to diblock copolymer (TB:DB) is 45:55 w/w or more, preferably 47:53 w/w or more, more preferably 50:50 w/w or more.
28. The formulation according to any one of the preceding aspects, wherein Mₙ(B) is 650 to 3,200 g/mol; R(TB) is 2.5 to 7.5; Mₙ(C) is 200 to 3,000 g/mol; and R(DB) is 1.5 to 11, preferably wherein:
(a) Mₙ(B) is 650 to 2000 g/mol; R(TB) is 2.5 to 5.5; Mₙ(C) is 1,000 to 3,000 g/mol; R(DB) is 1.5 to 4.5; the triblock copolymer (TB) content is 10 to 25% w/w, preferably 15 to 20% w/w, more preferably 15 to 18 or 18 to 20% w/w, relative to the total weight of the formulation; the diblock copolymer (DB) content is 10 to 25% w/w, preferably 15 to 20% w/w, more preferably 15 to 18 or 18 to 20% w/w, relative to the total weight of the formulation; the total content of polymers TB and DB is 20 to 50% w/w, preferably 30 to 40% w/w, more preferably 36 to 40% w/w; and the weight ratio of triblock to diblock copolymer (TB:DB) is 40:60 to 60:40 w/w; or
(b) Mₙ(B) is 650 to 2,000 g/mol; R(TB) 4.5 to 7.5; Mₙ(C) is 200 to 800 g/mol; R(DB) is 6.5 to 11; the triblock copolymer (TB) content is 15 to 25% w/w, relative to the total weight of the formulation; the diblock copolymer (DB) content is 15 to 25% w/w, relative to the total weight of the formulation; the total content of polymers TB and DB is 30 to 50% w/w; and the weight ratio of triblock to diblock copolymer (TB:DB) is 40:60 to 60:40 w/w; or
(c) Mₙ(B) is 1,500 to 3,200 g/mol; R(TB) is 2.5 to 5.5; Mₙ(C) is 1,000 to 3,000 g/mol; R(DB) is 1.5 to 4.5; the triblock copolymer (TB) content is 15 to 25% w/w, relative to the total weight of the formulation; the diblock copolymer (DB) content is 15 to 25% w/w, relative to the total weight of the formulation; the total content of polymers TB and DB is 30 to 50% w/w; and the weight ratio of triblock to diblock copolymer (TB:DB) is 40:60 to 60:40 w/w; or
(d) Mₙ(B) is 650 to 2,000 g/mol; R(TB) 4.5 to 7.5; Mₙ(C) is 200 to 800 g/mol; R(DB) is 6.5 to 11; the triblock copolymer (TB) content is 15 to 25% w/w, relative to the total weight of the formulation; the diblock copolymer (DB) content 10 to 25% w/w, relative to the total weight of the formulation; the total content of polymers TB and DB is 25 to 50% w/w; the weight ratio of triblock to diblock copolymer (TB:DB) is 40:60 to 60:40 w/w; or
(e) Mₙ(B) is 500 to 2,000 g/mol; R(TB) 4.5 to 7.5; Mₙ(C) is 200 to 800 g/mol; R(DB) is 6.5 to 11; the triblock copolymer (TB) content is 10 to 25% w/w, relative to the total weight of the formulation; the diblock copolymer (DB) content is 10 to 25% w/w, relative to the total weight of the formulation; the total content of polymers TB and DB is 20 to 50% w/w; the weight ratio of triblock to diblock copolymer (TB:DB) is 40:60 to 60:40 w/w; or
(f) Mₙ(B) is 500 to 2,000 g/mol; R(TB) 4.5 to 7.5; Mₙ(C) is 200 to 800 g/mol; R(DB) is 6.5 to 11; the triblock copolymer (TB) content is 10 to 25% w/w, relative to the total weight of the formulation; the diblock copolymer (DB) content is 15 to 25% w/w, relative to the total weight of the formulation; the total content of polymers TB and DB is 25 to 50% w/w; the weight ratio of triblock to diblock copolymer (TB:DB) is 40:60 to 60:40 w/w.
29. The formulation according to any one of the preceding aspects, wherein:
(a) Mₙ(B) is 800 to 1,800 g/mol; R(TB) is 3 to 5; Mₙ(C) is 1,500 to 2,500 g/mol; R(DB) is 2 to 4; the triblock copolymer (TB) content is 12 to 22% w/w, preferably 15 to 20% w/w, more preferably 15 to 18 or 18 to 20% w/w, relative to the total weight of the formulation; the diblock copolymer (DB) content is 12 to 22% w/w, preferably 15 to 20% w/w, more preferably 15 to 18 or 18 to 20% w/w, relative to the total weight of the formulation; the total content of polymers TB and DB is 24 to 44% w/w, preferably 30 to 40% w/w, more preferably 36 to 40% w/w; and the weight ratio of triblock to diblock copolymer (TB:DB) is 42:58 to 58:42 w/w; or
(b) Mₙ(B) is 800 to 1,800 g/mol; R(TB) 5 to 7; Mₙ(C) is 250 to 650 g/mol; R(DB) is 7.5 to 10.5; the triblock copolymer (TB) content is 18 to 22% w/w, relative to the total weight of the formulation; the diblock copolymer (DB) content is 18 to 22% w/w, relative to the total weight of the formulation; the total content of polymers TB and DB is 36 to 44% w/w; and the weight ratio of triblock to diblock copolymer (TB:DB) is 42:58 to 58:42 w/w; or
(c) Mₙ(B) is 1,800 to 3,200 g/mol; R(TB) is 3 to 5; Mₙ(C) is 1,500 to 2,500 g/mol; R(DB) is 2 to 4; the triblock copolymer (TB) content is 18 to 22% w/w, relative to the total weight of the formulation; the diblock copolymer (DB) content is 18 to 22% w/w, relative to the total weight of the formulation; the total content of polymers TB and DB is 36 to 44% w/w; and the weight ratio of triblock to diblock copolymer (TB:DB) is 42:58 to 58:42 w/w; or
(d) Mₙ(B) is 800 to 1,800 g/mol; R(TB) 5 to 7; Mₙ(C) is 250 to 650 g/mol; R(DB) is 7.5 to 10.5; the triblock copolymer (TB) content is 18 to 22% w/w, relative to the total weight of the formulation; the diblock copolymer (DB) content is 15 to 19% w/w, relative to the total weight of the formulation; the total content of polymers TB and DB is 33 to 41% w/w; the weight ratio of triblock to diblock copolymer (TB:DB) is 42:58 to 58:42 w/w; or
(e) Mₙ(B) is 800 to 1,800 g/mol; R(TB) 5 to 7; Mₙ(C) is 250 to 650 g/mol; R(DB) is 7.5 to 10.5; the triblock copolymer (TB) content is 15 to 19% w/w, relative to the total weight of the formulation; the diblock copolymer (DB) content is 17 to 21% w/w, relative to the total weight of the formulation; the total content of polymers TB and DB is 32 to 40% w/w; the weight ratio of triblock to diblock copolymer (TB:DB) is 42:58 to 58:42 w/w; or
(f) Mₙ(B) is 800 to 1,800 g/mol; R(TB) 5 to 7; Mₙ(C) is 250 to 650 g/mol; R(DB) is 7.5 to 10.5; the triblock copolymer (TB) content is 17 to 21% w/w, relative to the total weight of the formulation; the diblock copolymer (DB) content is 19 to 23% w/w, relative to the total weight of the formulation; the total content of polymers TB and DB is 36 to 44% w/w; the weight ratio of triblock to diblock copolymer (TB:DB) is 42:58 to 58:42 w/w.
30. The formulation according to any one of the preceding aspects, wherein the balance to 100% w/w is said water-miscible, pharmaceutically acceptable organic solvent and, optionally, one or more additional pharmaceutically acceptable excipient(s).
31. The formulation according to any one of the preceding aspects, which is in the form of an injectable liquid and/or is capable of passing through a filter with maximum pore size of 20 μm or less, preferably 20 μm or less and 0.2 μm or more, more preferably 0.2 μm.
32. Pharmaceutical dosage form comprising the formulation according to any one of aspects 1 to 31.
33. The dosage form according to aspect 32, wherein the total amount of etonogestrel per dosage unit is preferably 5 to 60 mg, more preferably 20 to 60 mg, most preferably 20 to 50 mg of etonogestrel.
34. The dosage form according to aspect 32 or 33, wherein the total amount of etonogestrel per dosage unit is 5 to 60 mg, preferably 10 to 60 mg, more preferably 15 to 60 mg, more preferably 20 to 60 mg, more preferably 35 to 60 mg, most preferably 20 to 60 mg of etonogestrel; and/or the volume of the formulation per dosage unit is 0.05 to 0.8 mL, preferably 0.1 to 0.8 mL, more preferably 0.3 to 0.8 mL.
35. The dosage form according to any one of aspects 32 to 34, wherein the volume of the formulation per dosage unit is preferably 0.05 to 0.8 mL, more preferably 0.2 to 0.8 mL, most preferably 0.2 to 0.6 mL.
36. The dosage form according to any one of aspects 32 to 35, wherein the total amount of etonogestrel per dosage unit is ≥50 mg, preferably 50 to 60 mg, more preferably >50 mg and ≤60 mg of etonogestrel; and/or the volume of the formulation per dosage unit is 0.5 to 1.5 mL, preferably 0.6 to 1.5 mL, more preferably 0.6 to 0.8 mL.
37. The dosage form according to any one of aspects 32 to 36, preferably 32, wherein the total amount of etonogestrel per dosage unit is 5 to 50 mg, preferably 10 to 50 mg, more preferably 15 to 45 mg, more preferably 20 to 45 mg, more preferably 35 to 45 mg, most preferably 20 to 40 mg of etonogestrel; and/or the volume of the formulation per dosage unit is 0.05 to 1.5 mL, preferably 0.05 to 0.5 mL, more preferably 0.1 to 0.5 mL, more preferably 0.1 to 0.3 mL, more preferably 0.3 to 0.5 mL, or most preferably 0.1 to 0.2 mL.
38. The formulation according to any one of aspects 1 to 31 and/or the dosage form according to any one of aspects 32 to 37, which is stable for at least 3 months, preferably 6 months, more preferably 12 months, further more preferably 18 months, most preferably 24 months, when stored at 30° C., or at 40° C. with a relative humidity of 75%.
39. The formulation according to any one of aspects 1 to 31 and/or the dosage form according to any one of aspects 32 to 37, which is stable for at least 3 months, preferably 6 months, more preferably 12 months, further more preferably 18 months, most preferably 24 months, when stored at 30° C.
40. The formulation according to any one of aspects 1 to 31 and/or the dosage form according to any one of aspects 32 to 37 or 39, which is stable for at least 3 months, preferably 6 months, more preferably 12 months, when stored at 40° C. with a relative humidity of 75%.
41. Method for preparing the formulation according to any one of aspects 1 to 31 and/or the dosage form according to any one of aspects 32 to 40, comprising the steps of:
(i) mixing:
   a triblock copolymer (TB) having the formula A-B-A′, wherein blocks A and A′ are formed of poly(D,L-lactic acid), and comprise, respectively, m and n lactic acid (LA) repeating units; block B is formed of poly(ethylene glycol) comprising p ethylene oxide (EO) repeating units; the molar ratio R(TB) of LA to EO repeating units (m+n)/p is 1.5 to 9;
   a diblock copolymer (DB) having the formula C-D, wherein block C is formed of methoxypoly(ethylene glycol) comprising r EO repeating units; block D is formed of poly(D,L-lactic acid) comprising q LA repeating units; and the molar ratio R(DB) of LA to EO repeating units (q/r) is 1.5 to 12; and
   a water-miscible, pharmaceutically acceptable organic solvent comprising DMSO, until the polymers are dissolved, thereby obtaining a vehicle; and
(ii) mixing the vehicle with etonogestrel, thereby obtaining the formulation;
(iii) optionally sterilizing the formulation; and
(iv) providing, and optionally packaging, a predetermined amount of the formulation, thereby obtaining the dosage form.
42. The method according to aspect 41, wherein:
   sterilizing in step (iii) comprises filtering the vehicle obtained in step (i) and/or the formulation obtained in step (ii) through a filter with maximum pore size of 20 μm or less, preferably 20 μm or less and 0.2 μm or more; more preferably 0.2 μm; and/or
   sterilizing in step (iii) comprises a step of dry heating the vehicle obtained in step (i) and/or the formulation obtained in step (ii); and/or
   mixing in step (i) and/or (ii) is performed by means of a roller mixer; and/or
   mixing in step (i) and/or (ii) is performed by means of an impellor reactor; and/or
   the method further comprises adding one or more additional pharmaceutically acceptable excipient(s) before, during or after step (i) and/or (ii).
43. Application device comprising the formulation or the dosage form according to any one of aspects 1 to 40, the device being adapted for administering the formulation or dosage form to a subject by injection, preferably subcutaneous or intramuscular injection, most preferably subcutaneous injection.
44. The application device according to aspect 43, which is a self-administration drug-delivery device or a syringe, preferably ready-to-use syringe.
45. The application device according to aspect 43 or 44, which is adapted for providing an injected volume of 0.05 to 1.5 mL, preferably 0.05 to 0.5 mL, more preferably 0.1 to 0.5 mL, more preferably 0.1 to 0.3 mL, more preferably 0.3 to 0.5 mL, most preferably 0.1 to 0.2 mL, of the formulation or dosage form.
46. The application device according to aspect 43 or 44, which is adapted for providing an injected volume of 0.05 to 0.8 mL, more preferably 0.2 to 0.8 mL, most preferably 0.2 to 0.6 mL.
47. Use of the formulation or the dosage form according to any one of aspects 1 to 40, or of the device according to any one of aspects 43 to 46, for contraception in a female subject.
48. The use according to aspect 47, comprising administration to the subject of a total amount of preferably 5 to 60 mg, more preferably 20 to 60 mg, most preferably 20 to 50 mg of etonogestrel per dose.
49. The use according to aspect 47 or 48, comprising administration to the subject of a total amount of 15 to 45 mg of etonogestrel per dose.
50. The use according to aspect 47 or 48, comprising administration to the subject of a total amount of ≥45 mg, preferably ≥50 mg, more preferably 45 to 60 mg, even more preferably 50 to 60 mg, most preferably >50 mg and ≤60 mg of etonogestrel per dose.
51. The use according to any one of aspects 47 to 50, comprising administration to the subject by injection, preferably subcutaneous or intramuscular injection, most preferably subcutaneous injection.
52. The use according to any one of aspects 47 to 51, wherein the time interval between two subsequent dose administrations is 90 days or more, preferably 120 days or more, more preferably 160 days or more.
53. The use according to any one of aspects 47 to 52, wherein the time interval between two subsequent dose administrations is 220 days or less, more preferably 160 to 220 days.
54. The use according to any one of aspects 47 to 53, wherein contraceptive effect is provided over a period of less than 3 years, preferably less than 2 years, more preferably less than 1 year after administration.

The present invention provides a formulation, as described above.

In one embodiment, block B has a number-average molecular weight (Mₙ(B)) of 500 to 3,500 g/mol; and/or block C has a number-average molecular weight (Mₙ(C)) of 175 to 3,000 g/mol. Number-average molecular weights can be determined by methods established in the art (as described further below).

In one embodiment, the triblock copolymer (TB) has a number-average molecular weight (Mₙ(TB)) of 1,700 to 48,000 g/mol, preferably 1,800 to 45,000 g/mol, more preferably 4,500 to 40,000 g/mol, more preferably 6,000 to 30,000 g/mol, more preferably 7,000 to 20,000 g/mol, more preferably 9,000 to 18,000 g/mol, more preferably 14,000 to 16,000 g/mol, most preferably 11,000 to 15,000 g/mol.

In one embodiment, the diblock copolymer (DB) has a number-average molecular weight (Mₙ(DB)) of 650 to 77,000 g/mol, preferably 1,000 to 50,000 g/mol, more preferably 2,000 to 30,000 g/mol, more preferably 3,000 to 15,000 g/mol, more preferably 4,000 to 11,000 g/mol, more preferably 6,000 to 12,000, more preferably 5,000 to 8,000 g/mol, more preferably 6,000 to 8,000 g/mol, or most preferably 5,500 to 7,500 g/mol.

In one embodiment, the triblock copolymer (TB) has a weight-average molecular weight (M_{w}(TB)) of 1,700 to 96,000 g/mol, preferably 1,800 to 90,000 g/mol, more preferably 4,500 to 80,000 g/mol, more preferably 6,000 to 60,000 g/mol, more preferably 7,000 to 40,000 g/mol, more preferably 9,000 to 36,000 g/mol, more preferably 14,000 to 32,000 g/mol; most preferably 11,000 to 30,000 g/mol.

In one embodiment, the diblock copolymer (DB) has a weight-average molecular weight (M_{w}(DB)) of 650 to 154,000 g/mol, preferably 1,000 to 100,000 g/mol, more preferably 2,000 to 60,000 g/mol, more preferably 3,000 to 30,000 g/mol, more preferably 4,000 to 22,000 g/mol, more preferably 6,000 to 24,000, more preferably 5,000 to 16,000 g/mol, more preferably 6,000 to 16,000, or most preferably 5,500 to 15,000 g/mol.

In one embodiment, Mₙ(B) is 500 to 3,500, preferably 650 to 3,000, more preferably 650 to 2,500, more preferably 800 to 2,200, more preferably 800 to 2,000, more preferably 950 to 1050, more preferably 1,000 to 2,000, most preferably 1,000 to 1,500 or 1,500 to 2,000 g/mol; and/or R(TB) is 1.5 to 8.0, preferably 2.5 to 8.0, more preferably 2.5 to 7.5, more preferably 3.5 to 7.5, more preferably 3.5 to 7.0, most preferably 4.5 to 7.5.

In one embodiment, Mₙ(C) is 175 to 3,000, preferably 200 to 3,000, more preferably 200 to 2,500, more preferably 250 to 2,500, more preferably 250 to 2,500, more preferably 350 to 2,000, more preferably 330 to 370, most preferably 350 to 1,200 or 1,200 to 2,000 g/mol; and/or R(DB) is 1.8 to 12.0, preferably 1.8 to 11.0, more preferably 2.5 to 11.0, more preferably 2.5 to 10.0, more preferably 3.0 to 10.0, more preferably 3.0 to 9.5, most preferably 6.5 to 10.5.

In one embodiment, Mₙ(B) is 500 to 2,000, preferably 600 to 1,800, more preferably 650 to 1,500, most preferably 800 to 1,200 g/mol; R(TB) is 1.5 to 6, preferably 2.5 to 6, more preferably 2.5 to 5, most preferably 3.5 to 4.5; Mₙ(C) is 700 to 3,000, preferably 700 to 2,500, more preferably 1,500 to 2,500, most preferably 1,800 to 2,200 g/mol; and R(DB) is 2 to 12, preferably 2.5 to 5, more preferably 2.5 to 3.5, most preferably 2.7 to 3.3.

In one embodiment, Mₙ(B) is 500 to 2,000, preferably 600 to 1,800, more preferably 650 to 1,500, most preferably 800 to 1,200 g/mol; R(TB) is 1.5 to 8, preferably 4 to 8, more preferably 4.5 to 7.5, most preferably 5.5 to 6.5; Mₙ(C) is 200 to 3,000, preferably 200 to 1,500, more preferably 250 to 1,500, most preferably 250 to 500 g/mol; and R(DB) is 2.5 to 12, preferably 2.5 to 11, more preferably 6.5 to 10.5, most preferably 7.5 to 9.5.

In one embodiment, Mₙ(B) is 1,200 to 3,200, preferably 1,200 to 2,500, more preferably 1,500 to 2,500, most preferably 1,800 to 2,200 g/mol; R(TB) is 1.5 to 8, preferably 5 to 8, more preferably 4 to 8, most preferably 3.2 to 3.8; Mₙ(C) is 175 to 2,500, preferably 250 to 2,500, more preferably 250 to 2,400, most preferably 300 to 2,400 g/mol; and R(DB) is 2 to 12, preferably 2.5 to 10, more preferably 2.5 to 10, most preferably 2.7 to 9.

In one embodiment, Mₙ(B) is 500 to 1,800, preferably 650 to 1,800, more preferably 650 to 1,500, most preferably 800 to 1,200 g/mol.

In one embodiment, Mₙ(C) is 200 to 1,500, preferably 200 to 500, most preferably 250 to 500 g/mol.

In one embodiment, the etonogestrel content is 2.5% w/w or more, or 15% w/w or less, preferably 2.5 to 15% w/w, more preferably 4 to 15% w/w, more preferably 4 to 12% w/w, more preferably 5 to 12% w/w, more preferably 5 to 10% w/w, more preferably 6 to 10% w/w, more preferably 6 to 10% w/w, most preferably 7 to 10% w/w, relative to the total weight of the formulation.

In one embodiment, the triblock copolymer (TB) content is 8% w/w or more, or 40% w/w or less, preferably 8 to 40% w/w, more preferably 9 to 40% w/w, more preferably 9 to 35% w/w, more preferably 10 to 35% w/w, more preferably 10 to 30% w/w, more preferably 15 to 30% w/w, more preferably 15 to 25% w/w, most preferably 17 to 21% w/w, relative to the total weight of the formulation.

In one embodiment, the diblock copolymer (DB) content is 6% w/w or more, or 50% w/w or less, preferably 6 to 50% w/w, preferably 10 to 50% w/w, more preferably 10 to 45% w/w, more preferably 15 to 40% w/w, more preferably 15 to 40% w/w, more preferably 17 to 40% w/w, more preferably 17 to 30% w/w, most preferably 17 to 21% w/w, relative to the total weight of the formulation.

In one embodiment, the total content of polymers TB and DB in the formulation is 14% w/w or more or 55% w/w or less, preferably 14 to 55% w/w, more preferably 18 to 55% w/w, more preferably 18 to 50% w/w, more preferably 30 to 55% w/w, more preferably 30 to 50% w/w, more preferably 32 to 50% w/w, more preferably 32 to 45% w/w, most preferably 34 to 42% w/w, relative to the total weight of the formulation. Alternatively, the total the total content of all polymers (i.e., including TB and DB) in the formulation is 14% w/w or more or 55% w/w or less, preferably 14 to 55% w/w, more preferably 18 to 55% 25 w/w, more preferably 18 to 50% w/w, more preferably 30 to 55% w/w, more preferably 30 to 50% w/w, more preferably 32 to 50% w/w, more preferably 32 to 45% w/w, most preferably 34 to 42% w/w, relative to the total weight of the formulation.

In one embodiment, the weight ratio of triblock to diblock copolymer (TB:DB) is 10:90 w/w or more, or 70:30 w/w or less, preferably 10:90 to 70:30 w/w, more preferably 10:90 to 65:35 w/w, more preferably 15:85 to 65:35 w/w, more preferably 15:85 to 60:40 w/w, more preferably 20:80 to 60:40 w/w, more preferably 20:80 to 55:45 w/w, more preferably 42:58 to 58:42 w/w, most preferably 47:53 to 53:47 w/w.

In one embodiment, the water-miscible, pharmaceutically acceptable organic solvent content is 40% w/w or more, or 82.5% w/w or less, preferably 45 to 82.5% w/w, more preferably 45 to 78% w/w, more preferably 40 to 78% w/w, more preferably 40 to 65% w/w, more preferably 45 to 65% w/w, more preferably 40 to 59% w/w, more preferably 46 to 57% w/w, most preferably 50 to 56% w/w, relative to the total weight of the formulation.

In one embodiment, the water-miscible, pharmaceutically acceptable organic solvent essentially consists of DMSO, or is a mixture of DMSO and one or more co-solvents, preferably N-methyl pyrrolidone (NMP); more preferably wherein the water-miscible, pharmaceutically acceptable organic solvent is DMSO.

In one embodiment, the formulation further comprises one or more additional pharmaceutically acceptable excipient(s), such as polylactic acid (PLA), polyethylene glycol or poly(lactic-co-glycolic acid) (PLGA).

In one embodiment, Mₙ(B) is 500 to 1,800, preferably 650 to 1,800, more preferably 650 to 1,500, most preferably 800 to 1,200 g/mol; R(TB) is 1.5 to 6, preferably 2.5 to 6, more preferably 2.5 to 5, most preferably 3.5 to 4.5; Mₙ(C) is 700 to 3,000, preferably 700 to 2,500, more preferably 1,500 to 2,500, most preferably 1,800 to 2,200 g/mol; and R(DB) is 2 to 12, preferably 2.5 to 5, more preferably 2.5 to 3.3, most preferably 2.7 to 3.3.

In one embodiment, Mₙ(B) is 500 to 1,800, preferably 650 to 1,800, more preferably 650 to 1,500, most preferably 800 to 1,200 g/mol; R(TB) is 1.5 to 8, preferably 2.5 to 8, more preferably 3.5 to 7.5, most preferably 4.5 to 7.5; Mₙ(C) is 175 to 3,000, preferably 200 to 1,500, more preferably 200 to 500, most preferably 250 to 500 g/mol; and R(DB) is 2.0 to 12.0, preferably 3.0 to 10.0, more preferably 3.0 to 9.5, most preferably 6.5 to 10.5.

In one embodiment, Mₙ(B) is 500 to 1,800, preferably 650 to 1,800, more preferably 650 to 1,500, most preferably 800 to 1,200 g/mol; R(TB) is 1.5 to 8, preferably 2.5 to 8, more preferably 3.5 to 7.5, most preferably 4.5 to 7.5; Mₙ(C) is 175 to 3,000, preferably 200 to 1,500, more preferably 200 to 500, most preferably 250 to 500 g/mol; and R(DB) is 2.0 to 12.0, preferably 3.0 to 10.0, more preferably 3.0 to 9.5, most preferably 6.5 to 10.5; and the etonogestrel content is 7% w/w or more, preferably 7.5% w/w or more, more preferably 8% w/w or more, more preferably 9% w/w or more, more preferably 10% w/w or more, relative to the total weight of the formulation.

In one embodiment, Mₙ(B) is 500 to 1,800, preferably 650 to 1,800, more preferably 650 to 1,500, most preferably 800 to 1,200 g/mol; R(TB) is 1.5 to 8, preferably 2.5 to 8, more preferably 3.5 to 7.5, most preferably 4.5 to 7.5; Mₙ(C) is 175 to 3,000, preferably 200 to 1,500, more preferably 200 to 500, most preferably 250 to 500 g/mol; and R(DB) is 2.0 to 12.0, preferably 3.0 to 10.0, more preferably 3.0 to 9.5, most preferably 6.5 to 10.5; and the etonogestrel content is 7% w/w or more and 10% w/w or less, preferably 7% w/w or more and 9% w/w or less, most preferably 7.5% w/w or more and 8% w/w or less, relative to the total weight of the formulation.

In one embodiment, the etonogestrel content is 7% w/w or more, preferably 7.5% w/w or more, more preferably 8% w/w or more, more preferably 9% w/w or more, more preferably 10% w/w or more, relative to the total weight of the formulation.

In one embodiment, the etonogestrel content is 7% w/w or more, preferably 7.5% w/w or more, preferably 8% w/w or more, more preferably 8% w/w or more, more preferably 9% w/w or more, more preferably 10% w/w or more, relative to the total weight of the formulation; and the water-miscible, pharmaceutically acceptable organic solvent content is 56% w/w or less, preferably 55% w/w or less, more preferably 54% w/w or less, relative to the total weight of the formulation.

In one embodiment, the etonogestrel content is 7% w/w or more, preferably 7.5% w/w or more, relative to the total weight of the formulation; and the water-miscible, pharmaceutically acceptable organic solvent content is 56% w/w or less, preferably 54% w/w or less, relative to the total weight of the formulation.

In one embodiment, the triblock copolymer (TB) content is 15% w/w or more, preferably 16% w/w or more, more preferably 17% w/w or more, relative to the total weight of the formulation; and the diblock copolymer (DB) content is 35% w/w or less, preferably 30% w/w or less, more preferably 25% w/w or less, relative to the total weight of the formulation; and the weight ratio of triblock to diblock copolymer (TB:DB) is 30:70 w/w or higher, preferably 40:60 w/w or higher, more preferably 50:50 w/w or higher.

In one embodiment, the total content of polymers TB and DB in the formulation is 30% w/w or more, preferably 32% w/w or more, more preferably 34% w/w or more, relative to the total weight of the formulation.

In one embodiment, the triblock copolymer (TB) content is 40% w/w or less, preferably 35% w/w or less, more preferably 30% w/w or less, relative to the total weight of the formulation; and the diblock copolymer (DB) content is 17% w/w or more, preferably 18.5% w/w or more, more preferably 20% w/w or more and 50% w/w or less, relative to the total weight of the formulation.

In one embodiment, the total content of polymers TB and DB in the formulation is 48% w/w or less, preferably 45% w/w or less, preferably 42% w/w or less, more preferably 40% w/w or less, relative to the total weight of the formulation; and the weight ratio of triblock to diblock copolymer (TB:DB) is 45:55 w/w or more, preferably 47:53 w/w or more, more preferably 50:50 w/w or more.

In one embodiment, the total content of polymers TB and DB in the formulation is 48% w/w or less, preferably 45% w/w or less, more preferably 40% w/w or less, relative to the total weight of the formulation; and the weight ratio of triblock to diblock copolymer (TB:DB) is 45:55 w/w or more, preferably 47:53 w/w or more, more preferably 50:50 w/w or more.

In one embodiment, Mₙ(B) is 650 to 3,200 g/mol; R(TB) is 2.5 to 7.5; Mₙ(C) is 200 to 3,000 g/mol; and R(DB) is 1.5 to 11.

In one embodiment, Mₙ(B) is 650 to 2,000 g/mol; R(TB) is 2.5 to 5.5; Mₙ(C) is 1,000 to 3,000 g/mol; R(DB) is 1.5 to 4.5; the triblock copolymer (TB) content is 10 to 25% w/w, preferably 15 to 20% w/w, more preferably 15 to 18 or 18 to 20% w/w, relative to the total weight of the formulation; the diblock copolymer (DB) content is 10 to 25% w/w, preferably 15 to 20% w/w, more preferably 15 to 18 or 18 to 20% w/w, relative to the total weight of the formulation; the total content of polymers TB and DB is 20 to 50% w/w, preferably 30 to 40% w/w, more preferably 36 to 40% w/w; and the weight ratio of triblock to diblock copolymer (TB:DB) is 40:60 to 60:40 w/w.

In one embodiment, Mₙ(B) is 650 to 2,000 g/mol; R(TB) 4.5 to 7.5; Mₙ(C) is 200 to 800 g/mol; R(DB) is 6.5 to 11; the triblock copolymer (TB) content is 15 to 25% w/w, relative to the total weight of the formulation; the diblock copolymer (DB) content is 15 to 25% w/w, relative to the total weight of the formulation; the total content of polymers TB and DB is 30 to 50% w/w; and the weight ratio of triblock to diblock copolymer (TB:DB) is 40:60 to 60:40 w/w.

In one embodiment, Mₙ(B) is 1,500 to 3,200 g/mol; R(TB) is 2.5 to 5.5; Mₙ(C) is 1,000 to 3,000 g/mol; R(DB) is 1.5 to 4.5; the triblock copolymer (TB) content is 15 to 25% w/w, relative to the total weight of the formulation; the diblock copolymer (DB) content is 15 to 25% w/w, relative to the total weight of the formulation; the total content of polymers TB and DB is 30 to 50% w/w; and the weight ratio of triblock to diblock copolymer (TB:DB) is 40:60 to 60:40 w/w.

In one embodiment, Mₙ(B) is 650 to 2,000 g/mol; R(TB) 4.5 to 7.5; Mₙ(C) is 200 to 800 g/mol; R(DB) is 6.5 to 11; the triblock copolymer (TB) content is 15 to 25% w/w, relative to the total weight of the formulation; the diblock copolymer (DB) content 10 to 25% w/w, relative to the total weight of the formulation; the total content of polymers TB and DB is 25 to 50% w/w; the weight ratio of triblock to diblock copolymer (TB:DB) is 40:60 to 60:40 w/w.

In one embodiment, Mₙ(B) is 500 to 2,000 g/mol; R(TB) 4.5 to 7.5; Mₙ(C) is 200 to 800 g/mol; R(DB) is 6.5 to 11; the triblock copolymer (TB) content is 10 to 25% w/w, relative to the total weight of the formulation; the diblock copolymer (DB) content is 10 to 25% w/w, relative to the total weight of the formulation; the total content of polymers TB and DB is 20 to 50% w/w; the weight ratio of triblock to diblock copolymer (TB:DB) is 40:60 to 60:40 w/w.

In one embodiment, Mₙ(B) is 500 to 2,000 g/mol; R(TB) 4.5 to 7.5; Mₙ(C) is 200 to 800 g/mol; R(DB) is 6.5 to 11; the triblock copolymer (TB) content is 10 to 25% w/w, relative to the total weight of the formulation; the diblock copolymer (DB) content is 15 to 25% w/w, relative to the total weight of the formulation; the total content of polymers TB and DB is 25 to 50% w/w; the weight ratio of triblock to diblock copolymer (TB:DB) is 40:60 to 60:40 w/w.

In one embodiment, Mₙ(B) is 800 to 1,800 g/mol; R(TB) is 3 to 5; Mₙ(C) is 1,500 to 2,500 g/mol; R(DB) is 2 to 4; the triblock copolymer (TB) content is 12 to 22% w/w, preferably 15 to 20% w/w, more preferably 15 to 18 or 18 to 20% w/w, relative to the total weight of the formulation; the diblock copolymer (DB) content is 12 to 22% w/w, preferably 15 to 20% w/w, more preferably 15 to 18 or 18 to 20% w/w, relative to the total weight of the formulation; the total content of polymers TB and DB is 24 to 44% w/w, preferably 30 to 40% w/w, more preferably 36 to 40% w/w; and the weight ratio of triblock to diblock copolymer (TB:DB) is 42:58 to 58:42 w/w.

In one embodiment, Mₙ(B) is 800 to 1,800 g/mol; R(TB) 5 to 7; Mₙ(C) is 250 to 650 g/mol; R(DB) is 7.5 to 10.5; the triblock copolymer (TB) content is 18 to 22% w/w, relative to the total weight of the formulation; the diblock copolymer (DB) content is 18 to 22% w/w, relative to the total weight of the formulation; the total content of polymers TB and DB is 36 to 44% w/w; and the weight ratio of triblock to diblock copolymer (TB:DB) is 42:58 to 58:42 w/w.

In one embodiment, Mₙ(B) is 1,800 to 3,200 g/mol; R(TB) is 3 to 5; Mₙ(C) is 1,500 to 2,500 g/mol; R(DB) is 2 to 4; the triblock copolymer (TB) content is 18 to 22% w/w, relative to the total weight of the formulation; the diblock copolymer (DB) content is 18 to 22% w/w, relative to the total weight of the formulation; the total content of polymers TB and DB is 36 to 44% w/w; and the weight ratio of triblock to diblock copolymer (TB:DB) is 42:58 to 58:42 w/w.

In one embodiment, Mₙ(B) is 800 to 1,800 g/mol; R(TB) 5 to 7; Mₙ(C) is 250 to 650 g/mol; R(DB) is 7.5 to 10.5; the triblock copolymer (TB) content is 18 to 22% w/w, relative to the total weight of the formulation; the diblock copolymer (DB) content is 15 to 19% w/w, relative to the total weight of the formulation; the total content of polymers TB and DB is 33 to 41% w/w; the weight ratio of triblock to diblock copolymer (TB:DB) is 42:58 to 58:42 w/w.

In one embodiment, Mₙ(B) is 800 to 1,800 g/mol; R(TB) 5 to 7; Mₙ(C) is 250 to 650 g/mol; R(DB) is 7.5 to 10.5; the triblock copolymer (TB) content is 15 to 19% w/w, relative to the total weight of the formulation; the diblock copolymer (DB) content is 17 to 21% w/w, relative to the total weight of the formulation; the total content of polymers TB and DB is 32 to 40% w/w; the weight ratio of triblock to diblock copolymer (TB:DB) is 42:58 to 58:42 w/w.

In one embodiment, Mₙ(B) is 800 to 1,800 g/mol; R(TB) 5 to 7; Mₙ(C) is 250 to 650 g/mol; R(DB) is 7.5 to 10.5; the triblock copolymer (TB) content is 17 to 21% w/w, relative to the total weight of the formulation; the diblock copolymer (DB) content is 19 to 23% w/w, relative to the total weight of the formulation; the total content of polymers TB and DB is 36 to 44% w/w; the weight ratio of triblock to diblock copolymer (TB:DB) is 42:58 to 58:42 w/w.

In one embodiment, the formulation is composed as described in any of the preceding aspects or embodiments, and the balance to 100% w/w is said water-miscible, pharmaceutically acceptable organic solvent and, optionally, one or more additional pharmaceutically acceptable excipient(s).

In one embodiment, the formulation is in the form of an injectable liquid. The formulation is capable of passing through a filter, e.g., with maximum pore size of 20 μm or less, preferably 20 μm or less and 0.2 μm or more, more preferably 0.2 μm. Filtration can remove particles, such as impurities or microorganisms. 0.2 μm is generally considered as pore size suitable for obtaining a sterile filtrate.

The present invention also provides a pharmaceutical dosage form comprising the formulation, as described above. As used herein, a “dosage form” or “dosage unit” generally refers to an item comprising or consisting of a pre-defined amount of the formulation, such that a pre-defined amount of etonogestrel (dose) is contained therein. The dosage form can be, for example, a liquid for injection comprising or consisting of a pre-defined amount of the formulation; or a packaging (e.g., container such as an injection vial) comprising a pre-defined amount of the formulation.

In one embodiment, the total amount of etonogestrel per dosage unit is preferably 5 to 60 mg, more preferably 20 to 60 mg, most preferably 20 to 50 mg of etonogestrel.

In one embodiment, the total amount of etonogestrel per dosage unit is 5 to 60 mg, preferably 10 to 60 mg, more preferably 15 to 60 mg, more preferably 20 to 60 mg, more preferably 35 to 60 mg, most preferably 20 to 60 mg of etonogestrel; and/or the volume of the formulation per dosage unit is 0.05 to 0.8 mL, preferably 0.1 to 0.8 mL, more preferably 0.3 to 0.8 mL.

In one embodiment, the total amount of etonogestrel per dosage unit is 5 to 50 mg, preferably 10 to 50 mg, more preferably 15 to 45 mg, more preferably 20 to 45 mg, more preferably 35 to 45 mg, most preferably 20 to 40 mg of etonogestrel. Alternatively, the total amount of etonogestrel per dosage unit is ≥50 mg, preferably 50 to 60 mg, more preferably >50 mg and ≤60 mg of etonogestrel.

In one embodiment, the volume of the formulation per dosage unit is 0.05 to 1.5 mL, preferably 0.05 to 0.5 mL, more preferably 0.1 to 0.5 mL, more preferably 0.1 to 0.3 mL, more preferably 0.3 to 0.5 mL, or most preferably 0.1 to 0.2 mL. For example, for a suitable formulation be loaded with 7.5% etonogestrel, preferably 530 mg of formulation should be injected to reach a target dose of 40 mg, which is equivalent to approx. 450 to 500 μL (e.g., 460 μL). Alternatively, the volume of the formulation per dosage unit is 0.5 to 1.5 mL, preferably 0.6 to 1.5 mL, more preferably 0.6 to 0.8 mL. For example, for a suitable formulation to be loaded with 7.5% etonogestrel, preferably 795 mg of formulation should be injected to reach a target dose of 60 mg, which is equivalent to approx. 720 μL.

In one embodiment, the formulation or dosage form is stable for at least 3 months, preferably 6 months, more preferably 12 months, further more preferably 18 months, most preferably 24 months, when stored at 30° C., or at 40° C. with a relative humidity of 75%. Stability can be determined by various methods common in the art. For instance, the formulation or dosage form may be stable for at least 3 months, preferably 6 months, more preferably 12 months, further more preferably 18 months, most preferably 24 months, when stored at 30° C. For instance, the formulation or dosage form may be stable for at least 3 months, preferably 6 months, more preferably 12 months when stored at 40° C. with a relative humidity of 75%. Preferably, the formulation or dosage form may be considered stable over a given period if it fulfills, or substantially fulfils, one or more of the following criteria: etonogestrel assay (%): 100+/−5%; total related impurities (% area): ≤2%; dynamic viscosity (mPa·s): 10 viscosity+/−25%; injectability force (N): <25N.

The present invention also provides a method for preparing the formulation and/or dosage form as described above. When the method is performed up to and including step (ii), the formulation is obtained; when performed up to and including step (iii), a sterile formulation is obtained; when performed up to and including step (iv), the dosage form is obtained.

In one embodiment, the method for sterilization of step (iii) is a filtration through a filter with maximum pore size of 20 μm or less, preferably 20 μm or less and 0.2 μm or more; more preferably 0.2 μm.

In one embodiment, sterilizing in step (iii) comprises a step of dry heating the vehicle obtained in step (i) and/or the formulation obtained in step (ii).

In one embodiment, mixing in step (i) and/or (ii) is performed by means of a roller mixer.

In one embodiment, mixing in step (i) and/or (ii) is performed by means of an impellor reactor.

In one embodiment, the method further comprises adding one or more additional pharmaceutically acceptable excipient(s) as mentioned above before, during or after step (i) and/or (ii).

The present invention also provides an application device comprising the formulation or the dosage form, as described above, the device being adapted for administering the formulation or dosage form to a subject by injection, preferably subcutaneous or intramuscular injection, most preferably subcutaneous injection.

In one embodiment, the application device is a self-administration drug-delivery device or a syringe, preferably ready-to-use syringe.

In one embodiment, the application device is adapted for providing an injected volume of 0.05 to 1.5 mL, preferably 0.05 to 0.5 mL, more preferably 0.1 to 0.5 mL, more preferably 0.1 to 0.3 mL, more preferably 0.3 to 0.5 mL, most preferably 0.1 to 0.2 mL, of the formulation or dosage form. The application device may also be adapted for providing an injected volume of preferably 0.05 to 0.8 mL or 0.8 to 1.5 mL, more preferably 0.2 to 0.8 mL, most preferably 0.2 to 0.6 mL. In this context, “adapted for” means that the application device is capable of providing the previously mentioned pre-determined injected volume upon actuation. If the device has no or essentially no dead volume, it is considered to be adapted for providing the entire or essentially the entire volume of formulation or dosage form stored in it as an injected volume. Otherwise, the device is considered to be adapted for providing an injected volume equal to the entire volume of formulation or dosage form stored in it, minus the dead volume of the device.

The present invention also provides use of the formulation, dosage form or the device as described herein for contraception in a female subject. As used herein, “subject” generally refers to a mammal, preferably human.

In one embodiment, the use comprises administration to the subject of a total amount of preferably 5 to 60 mg, more preferably 20 to 60 mg, most preferably 20 to 50 mg of etonogestrel. In one embodiment, the use comprises administration to the subject of a total amount of 15 to 45 mg of etonogestrel per dose. That is, total amount of etonogestrel contained in the formulation/dosage form administered to the subject per single dose is 15 to 45 mg.

In one embodiment, the use comprises administration to the subject by injection, preferably subcutaneous or intramuscular injection, most preferably subcutaneous injection. In one embodiment, the time interval between two subsequent dose administrations is 90 days or more, preferably 120 days or more, more preferably 160 days or more, and/or 220 days or less, more preferably 160 to 220 days.

In one embodiment, contraceptive effect is provided over a period of less than 3 years, preferably less than 2 years, more preferably less than 1 year after administration.

Also disclosed herein is a method for contraception in a female subject, the method comprising administering an effective amount of the formulation or dosage form, preferably by means of the device as described herein, to a subject in need thereof. In one embodiment, the method comprises administration to the subject of a total amount of preferably 5 to 60 mg, more preferably 20 to 60 mg, most preferably 20 to 50 mg of etonogestrel per dose. In one embodiment, the method comprises administration to the subject of a total amount of 15 to 45 mg of etonogestrel per dose; in an alternative embodiment, the method comprises administration to the subject of a total amount of ≥45 mg, preferably ≥50 mg, more preferably 45 to 60 mg, even more preferably 50 to 60 mg, most preferably >50 mg and ≤ 60 mg of etonogestrel per dose. In one embodiment, the method comprises administration to the subject by injection, preferably subcutaneous or intramuscular injection. In one embodiment, the time interval between two subsequent dose administrations 120 days or more, preferably 160 days or more. In one embodiment, contraceptive effect is provided over a period of less than 3 years, preferably less than 2 years, more preferably less than 1 year after administration.

As used herein, the term “prodrug” refers to a pharmaceutically acceptable compound that can be converted to etonogestrel under physiological conditions (i.e., upon administration to a mammalian subject), or by solvolysis. Prodrugs are bioreversible derivatives of drug molecules that undergo an enzymatic and/or chemical transformation in vivo to release the active parent drug, which can then exert the desired pharmacological effect (see, e.g., Rautio, J. et al. Prodrugs: design and clinical applications. Nat Rev Drug Discov 7, 255-270 (2008)). The term “prodrug” is also meant to include any covalently bonded carriers, which release etonogestrel in vivo when administered to a mammalian subject. Preferably, a prodrug is converted in vivo to etonogestrel, for example, by hydrolysis or oxidation reactions. Prodrugs can include: compounds wherein a hydroxy group of etonogestrel is bonded to any group that cleaves to form a free hydroxy group when the prodrug is administered to a mammalian subject, such as ester derivatives (such as alkyl esters, fatty esters, phenoxyacetate, or sulfate) or acetal derivatives (such as glucuronide) of a hydroxy functional group; and compounds which are transformed by oxidation to etonogestrel when administered to a mammalian subject (such as desogestrel).

The etonogestrel content/amount, as used herein, is always expressed based on the parent drug molecule (C₂₂H₂₈O₂; M=324.5 g/mol). If prodrugs or salts are present or used, their actual content/amount is recalculated in accordance to ratio between their respective molar weight and the molar weight of pure etonogestrel.

Generally, Mₙ(B) can be calculated as follows: Mₙ(B)=Mₙ(TB)/(1+R(TB)*72/44), wherein 72 and 44 correspond, respectively, to the unit molar weights of the LA and EO units; R(TB) is the molar ratio of LA to EO repeating units in the triblock copolymer, e.g., determined by NMR (as described below); and Mₙ(TB) is the number-average molecular weight of the triblock copolymer, e.g., determined by GPC (as described below); Mₙ(B) as used herein is preferably defined in this way.

Alternatively, Mₙ(TB) can be calculated as follows: Mₙ(TB)=Mₙ(B)*(1+R(TB)*72/44), wherein Mₙ(B) is the number-average molecular weight, e.g., determined by GPCC (as described below), of the PEG monoblock polymer (i.e., poly(ethylene glycol)) used as a starting material in the synthesis of the triblock copolymer (TB); and R(TB) is the molar ratio of LA to EO repeating units in the triblock copolymer, e.g., determined by NMR (as described below).

Generally, Mₙ(C) can be calculated as follows: Mₙ(C)=Mₙ(DB)/(1+R(DB)*72/44), wherein 72 and 44 correspond, respectively, to the unit molar weights of the LA and EO units; R(DB) is the molar ratio of LA to EO repeating units in the diblock copolymer, e.g., determined by NMR (as described below); and Mₙ(DB) is the number-average molecular weight of the diblock copolymer, e.g., determined by GPC C (as described below); Mₙ(C) as used herein is preferably defined in this way.

Alternatively, Mₙ(DB) can be calculated as follows: Mₙ(DB)=Mₙ(C)*(1+R(DB)*72/44), wherein Mₙ(C) is the number-average molecular weight, e.g., determined by GPC (as described below), of the of the mPEG monoblock polymer (i.e., methoxypoly(ethylene glycol)) used as a starting material in the synthesis of the diblock copolymer (DB); and R(DB) is the molar ratio of LA to EO repeating units in the diblock copolymer, e.g., determined by NMR (as described below).

As used herein, the number-average molecular weight (Mₙ) can be determined by methods known in the art, e.g., by GPC in THF at 25 or 35° C. As used herein, the weight-average molecular weight (M_{w}) can be determined by methods known in the art, e.g., by GPC in THF at 25 or 35° C.

Preferably, GPC analyses can be performed using a Waters Alliance HPLC e2695 instrument with a refractive index detector. The equipment can be equipped with a series of Waters styragel columns HR4, HR3 and HR2 kept at 35° C. The sample are run in BHT-stabilized THF at a constant flow rate of 1 mL/min. Molecular weights distributions are determined by conventional calibration using polystyrene calibration standards obtained from Waters.

As used herein, the R ratio, which describes the ratio between lactic acid units over ethylene oxide units (LA/EO), can be determined by ¹H NMR spectroscopy (e.g., on a 300 MHz spectrometer) wherein chemical shifts are referenced to the δ=7.26 ppm solvent value of CDCl₃. For this, all peaks are integrated separately. The intensity of the signal (integration value) is directly proportional to the number of hydrogens that constitutes the signal. To determine the R ratio (LA/EO ratio), the integration values need to be homogenous and representative of the same number of protons (e.g., all signal values are determined for ¹H). One characteristic peak of PLA and one of PEG are then used to determine the LA/EO ratio.

### EXAMPLES

### Example 1: Synthesis of Copolymers and Preparation of Formulations

### Linear Block Copolymers Synthesis

Copolymers were synthesized according to the method described in the U.S. Pat. No. 6,350,812, with minor modifications. Typically, the necessary amount of PEG (gives the triblock co-polymer) or methoxy-PEG (gives the diblock copolymer) was heated at 65° C., and dried under vacuum for 2 hours in a reactor vessel. DL-lactide (corresponding to the targeted LA/EO molar ratio) and zinc lactate ( 1/1000 of amount of lactide) were added. The reaction mixture was first dehydrated by three short vacuum/N₂ cycles. The reaction mixture was heated at 140° C., and rapidly degassed under vacuum. The reaction was conducted for four days at 140° C. under constant nitrogen flow (0.2 bar). The reaction was cooled to room temperature, and its content was dissolved in acetone and then subjected to precipitation with ethanol. The product obtained was subsequently dried under reduced pressure.

The product obtained was characterized by GPC for its Mₙ and its dispersity (D) determination, and by ¹H NMR for its residual lactide content and for the determination of the R ratio.

¹H NMR spectroscopy was performed using a Brucker Advance 300 MHz spectrometer. For all ¹H NMR spectrograms, TopSpin software was used for the integration of peaks and their analyses. Chemical shifts were referenced to the δ=7.26 ppm solvent value of CDCl₃.

For the determination of the R ratio, which describes the ratio between lactic acid units over ethylene oxide units (LA/EO), all peaks were integrated separately. The intensity of the signal (integration value) is directly proportional to the number of hydrogens that constitutes the signal. To determine the R ratio (LA/EO ratio), the integration values need to be homogenous and representative of the same number of protons (e.g. all signal values are determined for ¹H). One characteristic peak of PLA, and one of PEG are then used to determine the LA/EO ratio. This method is particularly suitable for molecular weights of PEGs above 1000 g/mol where the signal obtained for the polymer end-functions can be neglected.

GPC analyses were performed using a Waters Alliance HPLC e2695 instrument with a refractive index detector. The equipment was equipped with a series of waters styragel columns HR4, HR3, and HR2 kept at 35° C. The sample were run in BHT-stabilized THF at a constant flow rate of 1 mL/min. Molecular weights distributions were determined by conventional calibration using polystyrene calibration standards obtained from Waters.

### Formulation Preparation

Typically, sustained release formulations were prepared as follows: In an empty and tared glass vial, required copolymer amounts were weighed. The glass vial was tared again. An accurate DMSO mass was added. Vehicles (copolymer+solvent) were then placed on a roller mixer at room temperature (RT) overnight until complete copolymer dissolution. The required etonogestrel amount was weighed in a separate glass vial, and vehicle was carefully transferred on top of it. The vial was then placed on a roller mixer at least 24 hours at RT. The headspace of each vial was flushed with constant nitrogen flush for 30 seconds.

The formulations shown in Table 1 were prepared according to the above general procedures. The dose indicated indicates that an amount of the respective formulation containing the dose noted in the Table was used as the dosage form in the subsequent in vitro release or pharmacokinetic experiments.

TABLE 1
Summary of formulation characteristics

| | | Total | | | | | | | | |
|---|---|---|---|---|---|---|---|---|---|---|
| Formulation | Etonogestrel | polymer | DMSO | Mₙ(DB) | | DB | Mₙ(TB) | | TB | Dose |
| # | (w/w %) | (w/w %) | (w/w %) | (kg/mol) | R(DB) | (w/w %) | (kg/mol) | R(TB) | (w/w %) | (mg) |
| #1 | 1.5 | 40 | 58.5 | 8 | 10.1 | 40 | NA | NA | 0 | 10 |
| #2 | 1.5 | 40 | 58.5 | NA | NA | 0 | 9 | 3.7 | 40 | 10 |
| #3 | 5 | 50 | 45 | 8 | 10.1 | 25 | 11 | 5.8 | 25 | 10 |
| #4 | 10 | 40 | 50 | 10 | 2.8 | 20 | 9 | 3.7 | 20 | 10 |
| #5 | 10 | 40 | 50 | 10 | 2.8 | 20 | 18 | 3.3 | 20 | 10 |
| #6 | 5 | 40 | 55 | 10 | 2.8 | 20 | 9 | 3.7 | 20 | 10 |
| #7 | 10 | 30 | 60 | 10 | 2.8 | 15 | 9 | 3.7 | 15 | 10 |
| #8 | 7.5 | 40 | 52.5 | 7 | 8.3 | 20 | 15 | 5.4 | 20 | 10 |
| #9 | 7.5 | 40 | 52.5 | 7 | 8.3 | 20 | 15 | 5.4 | 20 | 15 |
| #10 | 7.5 | 40 | 52.5 | 7 | 8.3 | 20 | 15 | 5.4 | 20 | 20 |
| #11 | 7.5 | 40 | 52.5 | 7 | 8.3 | 20 | 15 | 5.4 | 20 | 40 |
| #12 | 10 | 34 | 56 | 7 | 8.3 | 17 | 15 | 5.4 | 17 | 15 |
| #13 | 9 | 38 | 53 | 7 | 8.3 | 19 | 15 | 5.4 | 19 | 15 |
| #14 | 8 | 42 | 50 | 7 | 8.3 | 21 | 15 | 5.4 | 21 | 15 |
| #15 | 10 | 34 | 56 | 6 | 9.2 | 17 | 15 | 6.6 | 17 | 40 |
| #16 | 9 | 38 | 53 | 6 | 9.2 | 19 | 15 | 6.6 | 19 | 40 |
| #17 | 8 | 42 | 50 | 6 | 9.2 | 21 | 15 | 6.6 | 21 | 40 |
| #18 | 7.5 | 40 | 52.5 | 6 | 9.2 | 20 | 15 | 6.6 | 20 | 40 |

NA: Not applicable

### Example 2: In Vitro Release (IVR) Tests

Typically, a formulation mass equivalent to a dose of 10 to 50 mg of etonogestrel was injected into a 150 mL Erlenmeyer containing 50 mL of Phosphate buffer pH 7.4 (PBS)+0.5% (w/V) of hydroxypropyl-β-cyclodextrin (HPBCD). The Erlenmeyer was then placed in a climatic chamber at 37° C. on an orbital shaker at 65 rpm. At pre-determined time intervals, around 2 mL of medium were collected, and filtered through a 0.2 μm hydrophilic filter into a 1.5 mL HPLC glass vial before analysis by UPLC; the rest of the medium was discarded, and fresh buffer was added to the Erlenmeyer which was then placed back on orbital shaker at 37° C. Sink conditions were maintained during the full duration of the study.

The amount of API released from the depot was determined using a Waters Acquity UPLC system with a UV detector set at 244 nm and a C18 analytical column (BEH C18, Waters). The temperature of the column was maintained at 48° C., and the flow rate fixed at 0.3 mL/min. Water/formic acid (100:0.1 V/V; solvent A) and acetonitrile/formic acid (100:0.1 V/V; solvent B) were used as mobile phases with an initial 50/50 composition. The proportion of solvent B was increased to 95% from 1 to 1.5 min before a wash step of 0.5 min, and a reequilibration period to 50/50 of 2.5 min.

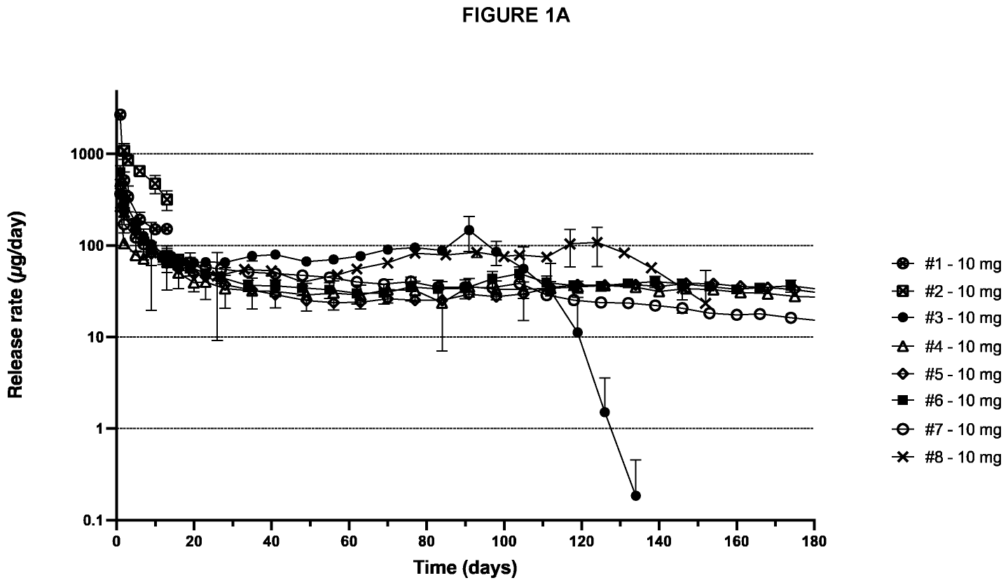

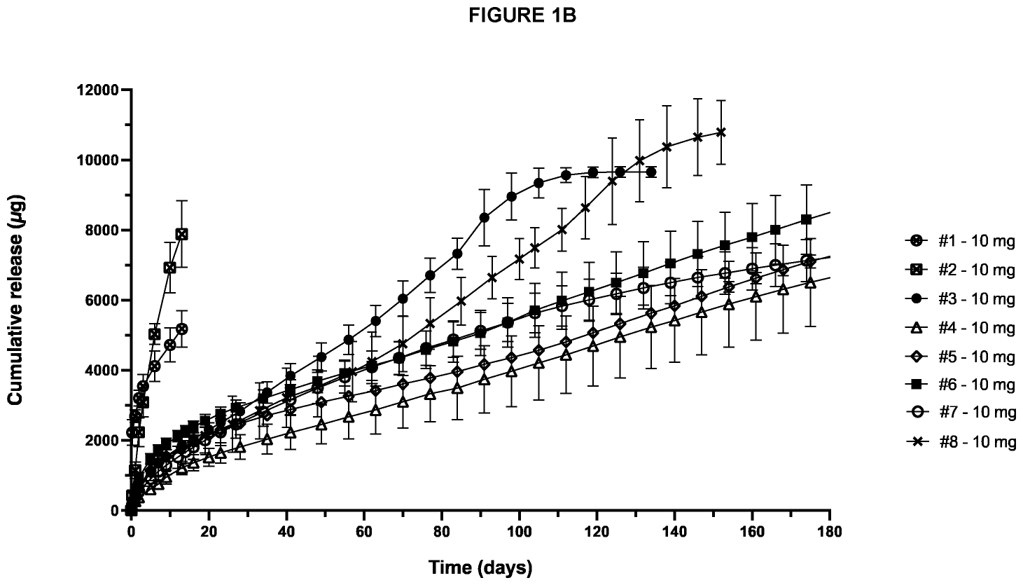

The results are shown in FIGS. 1 to 3.

As shown in FIGS. 1A and 1B, while keeping the etonogestrel dose constant, by modifying the component of the formulations and their contents, a wide range of release profiles can be obtained in vitro. In particular, after an initial burst, a plateau can be reached for several weeks, with a constant quantity of API released per day.

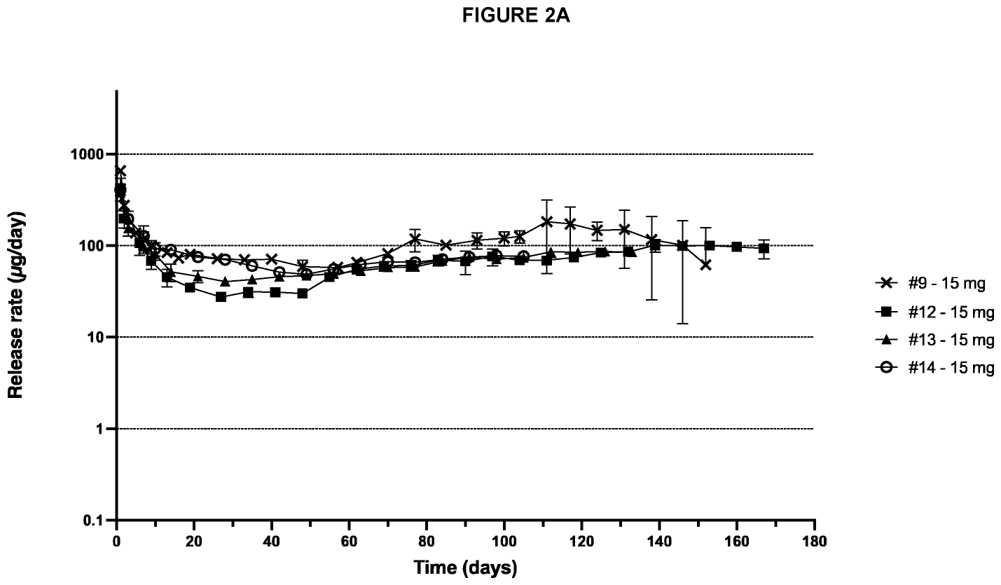

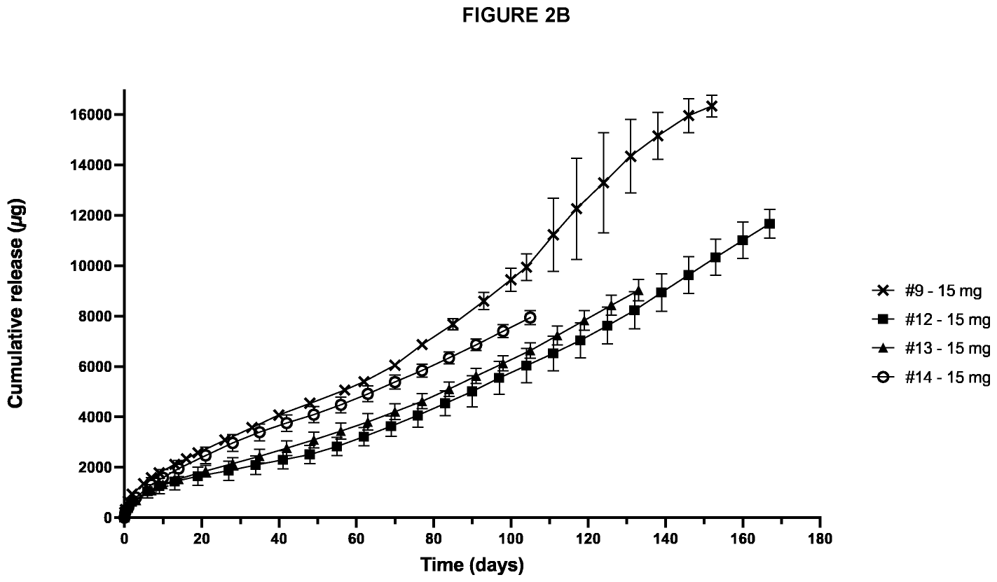

FIGS. 2A and 2B show that by keeping the etonogestrel dose and the components of the formulation fixed, a modulation of the etonogestrel release can still be obtained when modifying the contents of the components.

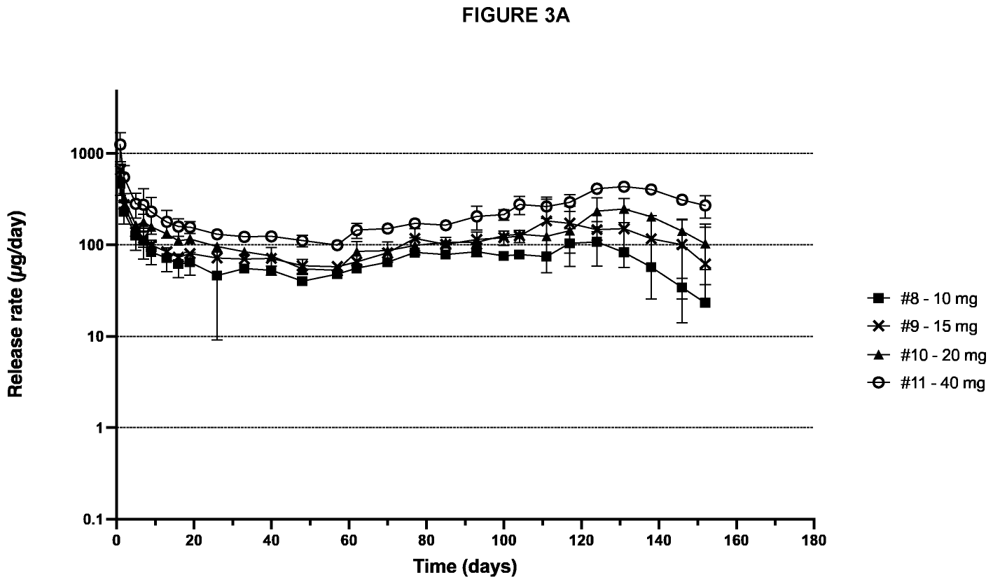

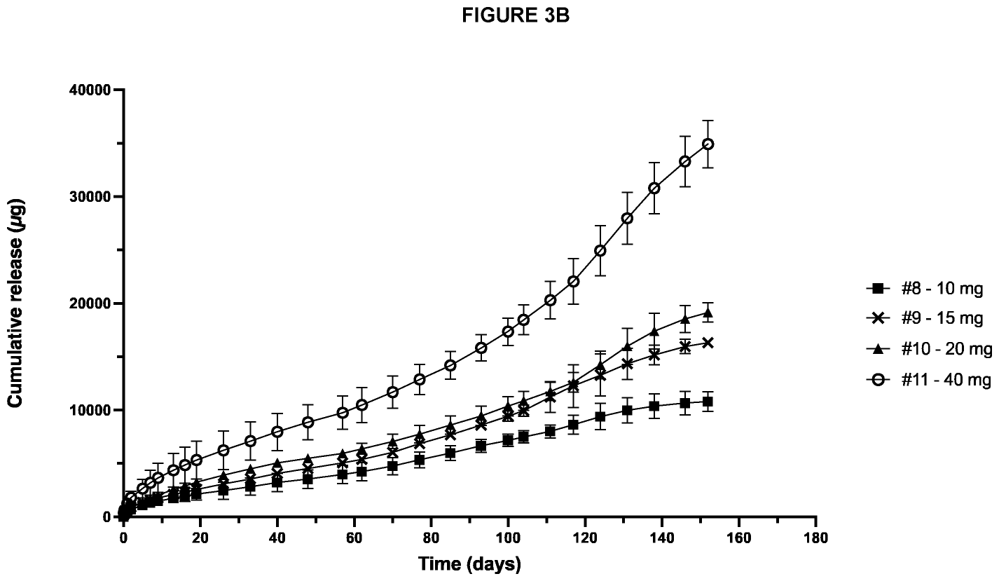

The impact of the etonogestrel dose is presented on FIGS. 3A and 3B. It can be observed that at a fixed composition, different release profiles of etonogestrel can still be obtained by changing the formulation volume, and thus the API dose.

### Example 3: Pharmacokinetic (PK) Studies in Dogs

### In Vivo Detailed Set-Up Procedure

Three etonogestrel formulations (#3, #7 and #9) were tested in pharmacokinetic studies in female Beagle dogs of 10-12 months old of approximately 7 to 12 kg. Drug products containing 10 mg (#3 and #7) or 15 mg (#9) of Etonogestrel were subcutaneously administered in the interscapular area of the dogs using syringes and 21 G ⅝-inch needles. Injected formulations volumes were fixed at 90 μL, 170 μL or 180 μL depending on the initial Etonogestrel content in the formulations. Number of dogs per group was fixed at 5.

Animal observations were recorded after administration and at least once a day throughout the study and injection sites were observed at each PK time-point. Blood samples were collected into K₂-EDTA anticoagulant tubes at predose and at different time points post-dose: T0.5 h, T1 h, T2 h, T3 h, T6 h, T10 h, D1 (day 1), D2, D4, D7, D14, D21, D28 (1 month), D42, D56 (2 months), D70, D84 (3 months), D98, D112 (4 months), D126, D140 (5 months), D154, D168 (6 months), D182, D196 (7 months) and D210. Blood samples were centrifuged, and plasma were separated and analyzed to determine the plasmatic concentration of Etonogestrel using a qualified analytical procedure.

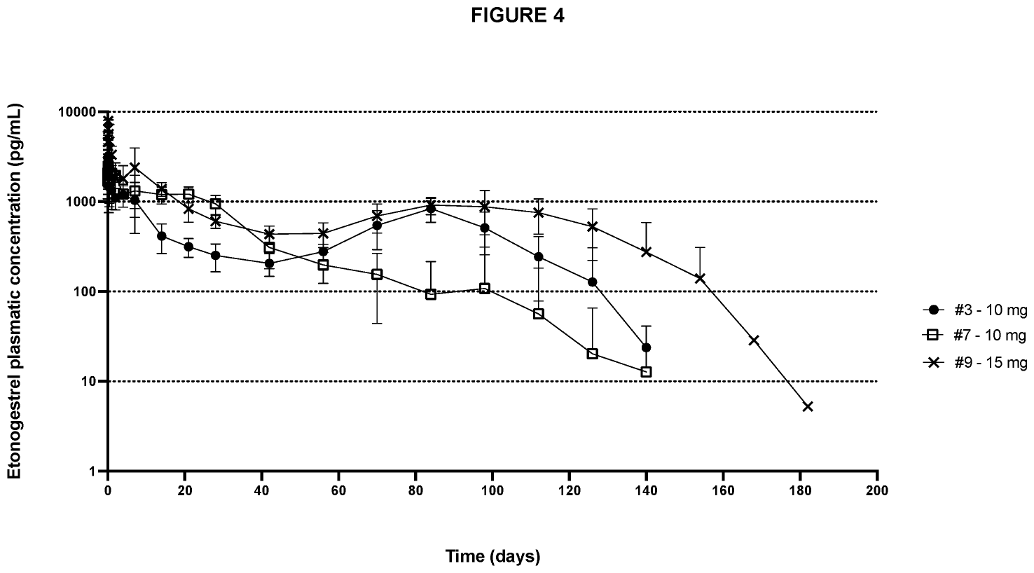

The results are shown in FIG. 4; main PK parameters are summarized in Table 2.

TABLE 2
Summary of main PK parameters [Mean (CV %)]

| Test | Tₘₐₓ ⁽¹⁾ | Tₗₐₛₜ ⁽¹⁾ | Cₘₐₓ | Cₘₐₓ/D ⁽²⁾ | AUCₗₐₛₜ | AUCₗₐₛₜ/D ⁽²⁾ | T_{1/2} ⁽³⁾ |
|---|---|---|---|---|---|---|---|
| item | (Day) | (Day) | (pg/mL) | (pg/mL/mg) | (day*pg/mL) | (day*pg/mL/mg) | (Day) |
| #3 | 0.13 | 126.1 | 3341 | 333.5 | 56460 | 5650 | 11.5* |
| | [0.08-0.13] | [97.98-140.1] | (30.0) | (29.5) | (9.1) | (10.0) | |
| #7 | 0.13 | 84.03 | 3039 | 335.2 | 53830 | 5908 | 15.1 |
| | [0.08-2] | [69.99-1401.1] | (21.0) | (22.8) | (7.9) | (5.3) | |
| #9 | 0.125 | 154.0 | 8422 | 556.1 | 115900 | 7612 | 8.0 |
| | [0.021-0.125] | [126.0-182.0] | (28) | (33) | (9) | (12) | |

⁽¹⁾ Median [min-max]
⁽²⁾ Corrected by the actual dose
⁽³⁾ Harmonic mean
*N = 2 for this value

Data show that quantifiable etonogestrel level can be measured for up to 182 days. The release duration depends on formulation composition and dose. For the 3 tested formulations, the Cₘₐₓ was rapidly reached, 3 hours after formulation injection, and followed by a reduction of plasma concentrations. For #3 and #9 a re-increased of API plasmatic level is observed from day 42 to day 84, while #7 induces a continuously decreasing level.

Similarly, other etonogestrel formulations (#15, #16, #17 and #18) were tested in a pharmacokinetic study in female Beagle dogs of at least 7 months old and of approximately 6 to 9 kg. Drug products containing 40 mg of Etonogestrel were subcutaneously administered in the interscapular area of the dogs using syringes and 21 G ⅝-inch needles. Injected formulations volumes, depending on the initial Etonogestrel concentration in each formulation, varied between 350 UL and 480 μL per dog. Number of dogs per group was fixed at 5.

Animal observations were recorded after administration and at least once a day throughout the study, and injection sites were observed twice a week for the first month and then at each PK time-point. Blood samples were collected into K₂-EDTA anticoagulant tubes at predose and at different time points post-dose selected from: T0.5 h, T1 h, T2 h, T3 h, T6 h, T10 h, 24 h (D1; day 1), D2, D4, D7, D14, D21, D28 (1 month), D42, D56 (2 months), D70, D84 (3 months), D98, D112 (4 months), D126, D140 (5 months), D154, D168 (6 months), D182, D196 (7 months), D210 and D224. Blood samples were centrifuged and plasma was separated and analyzed to determine the plasmatic concentration of Etonogestrel using a qualified analytical procedure.

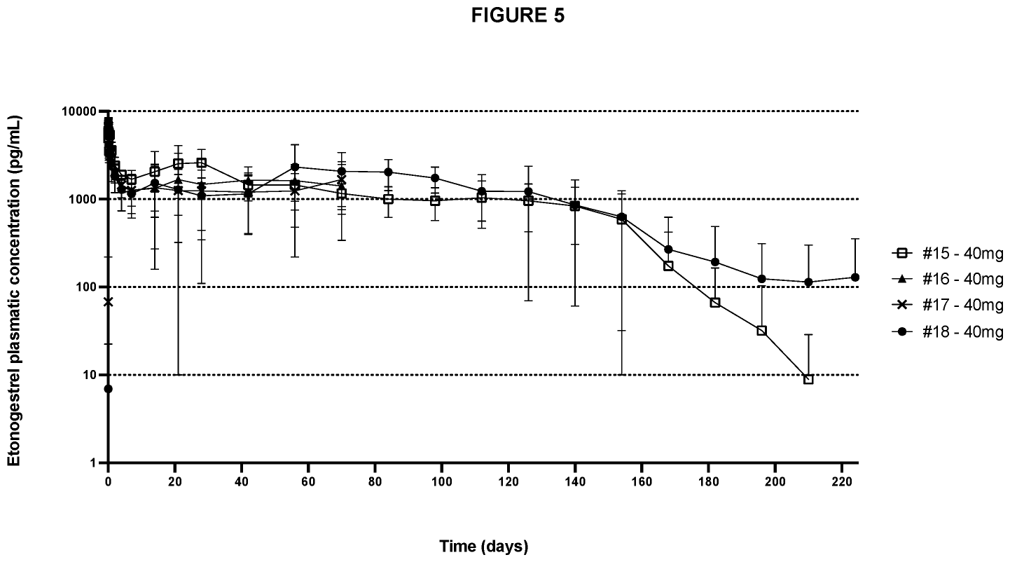

The results are shown in FIG. 5; main PK parameters are summarized in Table 3.

TABLE 3
Summary of main PK parameters [Mean (CV %)]

| Test | Tₘₐₓ ⁽¹⁾ | Tₗₐₛₜ ⁽¹⁾ | Cₘₐₓ | Cₘₐₓ/D ⁽²⁾ | AUC_{0-70 d}* | AUC_{0-70 d}/D ⁽²⁾* | AUCₗₐₛₜ | AUCₗₐₛₜ/D ⁽²⁾ | T_{1/2} ⁽³⁾ |
|---|---|---|---|---|---|---|---|---|---|
| item | (Day) | (Day) | (pg/mL) | (pg/mL/mg) | (day*pg/mL) | (day*pg/mL/mg) | (day*pg/mL) | (day*pg/mL/mg) | (Day) |
| #15 | 0.13 | 154 | 6245 | 153.3 | 129800 | 3194 | 214200 | 5264 | 15.1 |
| | [0.04-0.42] | [154-210] | (11.4) | (12.3) | (21.3) | (22.6) | (10.7) | (12.3) | |
| #16 | 0.08 | 70 | 7149 | 179.5 | 109900 | 2746 | NR | NR | NR |
| | [0.02-0.13] | [70-70] | (23.5) | (24.5) | (41.1) | (40.9) | | | |
| #17 | 0.04 | 70 | 7030 | 174.5 | 95550 | 2358 | NR | NR | NR |
| | [0.02-0.13] | [70-70] | (23.7) | (22.6) | (59.4) | (57.9) | | | |
| #18 | 0.13 | 168 | 7205 | 173.5 | 109600 | 2636 | 239900 | 5769 | 10.8 |
| | [0.08-0.13] | [126-224] | (7.2) | (7.8) | (30.2) | (29.8) | (12.7) | (11.7) | |

⁽¹⁾ Median [min-max]
⁽²⁾ Corrected by the actual dose
⁽³⁾ Harmonic mean
NR: No result
*AUC_{0-70 d} was calculated to do comparison between all groups as treated groups #16 and #17 were stopped at Day 70.

Intermediate results over 28 days were used to compare the peak level of the 4 test items. The data showed similar plasma profiles whatever the test items, with #15 provided a very slight lower Cmax.

The entire data set showed that etonogestrel levels of individual animals can be quantified over 154-210 days and 126-224 days with #15 and #18, respectively. The behaviour of #15 and #18 was quite similar during the 3 first hours with a rapid increase of concentrations (up to Cmax) followed by a drop of plasma concentrations. Then two bumps (i.e. re-increase in plasma levels) were observed for each formulation.

Comparing #18 tested in this last study at 40 mg with #9 tested in the previous study at 15 mg in dogs showed that the highest dose of 40 mg increased the overall plasma exposure (AUC) but did not provide higher peak level of etonogestrel compared to 15 mg, which is considered as an important point for safety purpose.

### Example 4: Stability Study

The stability of 800 mg aliquots of composition as in formulation #3 was evaluated in 3-ml vials under long-term stability condition in an oven at 30° C.±3° C. and under accelerated stability condition in an ICH climatic chamber at 40° C. with a relative humidity of 75%. The study was completed by the stability assessment of a second composition (as in formulation #8) under the same conditions, in an ICH climatic chamber at 25° C. with a relative humidity of 60% and under storage conditions of +2-8° C. Formulations were prepared as detailed in Example 2, and aliquoted in different nitrogen-flushed vials.

At study start and at different selected time-points over up to 24 months, etonogestrel and related substances contents were monitored by UPLC; water content within formulations was measured using a Karl Fischer volumetric titrator; copolymer integrity was checked by following molecular weight by GPC-RI and measuring sample dynamic viscosity and formulation injectability was determined. Detailed set-ups are described below. Further time-points are similarly assessed.

The amount of etonogestrel and related substances were determined using a Waters Acquity UPLC system with a UV detector set at 245 nm and a C18 analytical column (BEH C18, Waters). The temperature of the column was maintained at 30° C. and the flow rate fixed at 0.5 mL/min. Water/formic acid (100:0.1 V/V; solvent A) and acetonitrile/formic acid (100:0.1 V/V; solvent B) were used as mobile phases with an initial 81/19 composition. The proportion of solvent B was increased to 37% in 4 min, maintained constant for 3 min, increased to 75% in 3 min, and increased again to 95% in 0.5 min before a wash step of 1 min and a reequilibration period to 50/50 of 3.5 min.

For GPC-RI analyses, the needed formulation amount to reach a 10 mg/mL copolymer concentration was weighed into a 10 mL glass vial. The vial was then filled with BHT-stabilized THF, and left under orbital stirring. 1 mL of solution was then filtered through a 0.45 μm PTFE filter into an HPLC glass vial. Samples were then analyzed using method described in Example 1.

Dynamic viscosity analysis was performed using an Anton Paar Rheometer equipped with cone plate measuring system of 50 mm diameter and cone angle of 2 degrees. Temperature was fixed at 25° C. The formulation was vortexed for 30 s before analysis. 800 μL of formulation were placed at the center of the thermo-regulated measuring plate using a spatula. The measuring system was lowered down, and a 0.104 mm gap was left between the measuring system and the measuring plate. Twenty-one viscosity measurements points were determined across the 10 to 1000 s−1 shear rate (10 points per decade).

Injectability analyses were performed using a Lloyd Instruments FT plus texturometer fitted with Nexygen plus software. 500 μL of formulation were withdrawn from vial previously vortexed using a 1 mL Soft-ject syringe with a 19 G 1-inch needle. Air bubbles were removed to avoid any interference during the injectability measurement. 19 G needle was then replaced by a 21 G ⅝-inch Terumo needle. The syringe was placed onto the texturometer. The flow rate was fixed at 2 mL/min. Injection of the formulation started at fixed speed. The average force in Newton (N) necessary to inject each replicate was calculated using texturometer software. The injectability analyses were performed in triplicates.

Water content determination was performed using a V-30 titrator fitted with a Karl Fischer kit and equipped with an interchangeable burette and a sensor. 900 μL of formulation were withdrawn using a 1 ml syringe mounted with a 18 G 1-inch needle and approximately 300 UL of formulation were injected inside the titration cell. Exact formulation amount injected was determined by indirect weighing.

Results are presented in Tables 4 and 5.

All parameters remained consistent in the different storage conditions tested for the periods studied, showing that the formulations are stable over time under different storage conditions.

### Example 5: Sterilization Studies

Similar to the stability studies detailed in Example 4, the compatibility of dry heat sterilization method has been evaluated through the stability assessment of 800 mg aliquots of composition as in formulation #8 in 3 ml vials after dry heat cycles (+121° C./35 min and +111° C./60 min). Results are presented in Table 6.

All parameters remained consistent after dry heat cycles and after 7 months of storage at 40° C./75% RH, confirming the stability of the formulation after dry heat cycles, which is indicative for compatibility with dry heat sterilization.

Alternatively, the sterilization by filtration of etonogestrel formulations has also been evaluated with formulation batches of up to 5 L. Results showed that the filtration on 0.2 μm filter was possible with formulations of the invention.

TABLE 4
#3 stability study results over up to 24 months in different storage conditions

| | Study | Storage time at 30° C. (months) | | | | | | Storage time at 40° C./75% RH (months) | | |
|---|---|---|---|---|---|---|---|---|---|---|
| Test | start | 3 | 6 | 9 | 12 | 18 | 24 | 3 | 6 | 12 |
| Etonogestrel assay | NA | 101.2 | 99.5 | 98.3 | 99.7 | 97.1 | 98.9 | 101.3 | 98.2 | 99.1 |
| (% of t0) | | | | | | | | | | |
| Total related impurities | 0.0 | 0.1 | 0.3 | 0.3 | 0.2 | 0.4 | 0.3 | 0.4 | 0.2 | 0.3 |
| (% area) | | | | | | | | | | |
| Dynamic viscosity | 2421 | 2541 | 2609 | 2602 | 2541 | 2427 | 2542 | 2543 | 2570 | 2425 |
| (mPa · s) | | | | | | | | | | |
| Injectability force | 13.4 | 15.4 | 18.6 | 18.2 | 13.9 | 18.0 | 11.7 | 15.0 | 18.3 | 11.5 |
| (N) | | | | | | | | | | |
| GPC-RI (% Mn variation | NA | NT | NT | +1.4 | +1.6 | +0.7 | +1.2 | NT | NT | +0.2 |
| from t0) | | | | | | | | | | |
| Water content | NT | NT | NT | NT | 0.2 | 0.2 | 0.2 | NT | NT | 0.3 |
| (% w/w) | | | | | | | | | | |

NA: not applicable
NT: not tested

TABLE 5
#8 stability study results over 6 months in different storage conditions

| | | Storage time at +25° C./60% RH | | | Storage time at +30° C. | | | Storage time at +40° C./75% RH | | | |
|---|---|---|---|---|---|---|---|---|---|---|---|
| | Study | (months) | | | (months) | | | (months) | | | 6 months |
| Test | start | 1 | 3 | 6 | 1 | 3 | 6 | 1 | 3 | 6 | at +2-8° C. |
| Etonogestrel assay | 99.9 | 100.7 | 101.1 | 102.1 | 101.0 | 100.9 | 101.3 | 101.2 | 101.5 | 101.1 | 102.2 |
| (% LC) | | | | | | | | | | | |
| Total related impurities | <0.1 | <0.1 | <0.1 | 0.2 | <0.1 | <0.1 | 0.2 | <0.1 | <0.1 | 0.3 | <0.1 |
| (%) | | | | | | | | | | | |
| Dynamic viscosity | 771 | 761 | 750 | 754 | 767 | 748 | 745 | 770 | 748 | 741 | 760 |
| (mPa · s) | | | | | | | | | | | |
| Injectability force | 6.7 | 6.1 | 4.2 | 5.3 | 5.7 | 4.3 | 5.3 | 5.7 | 4.2 | 4.7 | 5.5 |
| (N) | | | | | | | | | | | |
| GPC-RI (% Mn variation | NA | +1.0 | +1.8 | +0.4 | +0.9 | +0.0 | +0.4 | +0.2 | +1.0 | −0.2 | +1.0 |
| from t0) | | | | | | | | | | | |
| Water content | 0.2 | 0.2 | 0.2 | 0.2 | 0.2 | 0.2 | 0.2 | 0.2 | 0.2 | 0.2 | 0.2 |
| (% w/w) | | | | | | | | | | | |

NA: not applicable
NT: not tested

TABLE 6
#8 7-month stability results after dry heat cycles

| | Study start | | | 3-month storage at +40° C./75% RH | | | 7-month storage at +40° C./75% RH | | |
|---|---|---|---|---|---|---|---|---|---|
| | | DH +121° | DH +111° | | DH +121° | DH +111° | | DH +121° | DH +111° |
| | Non- | C., 35 | C., 60 | Non- | C., 35 | C., 60 | Non- | C., 35 | C., 60 |
| Test | sterilized | min | min | sterilized | min | min | sterilized | min | min |
| Etonogestrel assay | 100.5 | 101.4 | 100.6 | 99.7 | 100.5 | 98.7 | 98.7 | 98.1 | 100.3 |
| (% LC) | | | | | | | | | |
| Total related impurities | 0.2 | 0.2 | 0.2 | 0.3 | 0.4 | 0.3 | 0.1 | 0.1 | 0.1 |
| (% area) | | | | | | | | | |
| Dynamic viscosity | 539 | 536 | 543 | 549 | 547 | 555 | 549 | 550 | 549 |
| (mPa · s) | | | | | | | | | |
| Injectability force | 4.7 | 4.7 | 5.2 | 5.1 | 5.3 | 4.5 | 4.7 | 4.7 | 5.2 |
| (N) | | | | | | | | | |
| GPC-RI | 8097 | 8071 | 8075 | NT | NT | NT | 8006 | 8040 | 8195 |
| (Mn g/mol) | | | | | | | | | |
| Water content (% w/w) | 0.1 | 0.1 | 0.1 | NT | NT | NT | 0.2 | 0.2 | 0.2 |

NT: not tested

### Example 6: Toxicology Study in Rats

The low systemic and local toxicity of a selected etonogestrel formulation and corresponding polymeric vehicle are confirmed by assessment performed on female rats (20 rats per group) of at least 13 week-old and weighing 200 to 300 g, after repeated subcutaneous administrations (4 administrations, 6 week-apart) for 26 weeks. This period of exposure is followed by a recovery period of about 4 weeks or more (10 rats per group), until estrous cycles return to normal in at least half of the animals in the high dose group.

Briefly, at D1, D43 (6 weeks), D85 (12 weeks) and D127 (18 weeks), each animal is injected a fixed dose (in mg/kg, based on the most recent body weight measurement) of either control saline solution, control polymeric vehicle or etonogestrel formulation, as detailed in Table 7. Injection is performed subcutaneously on 4 different sites for each animal. Throughout the study period, general in-life assessments (including mortality/morbidity, clinical observations, injection site observations, body weight measurement, food consumption monitoring) are made at predetermined timepoint. Moreover, the estrous cycles are monitored by vaginal smears sampling to determine the number and duration of cycles.

After 11, 25 and 30 weeks (and more when applicable), blood and urine samples are collected from each animal for clinical pathology (hematology, coagulation, clinical chemistry and urinalysis).

In addition, the toxicokinetic of the etonogestrel formulation are assessed over the study on dedicated animals. Blood samples for the determination of etonogestrel serum level are collected at predose and then at T0.5 h, T2 h, T3 h, T24 h, T168 h, and T672 h after each administration for the groups treated with the etonogestrel formulation (9 rats per group), and only until T2 h after administration for the group administered the control-treated groups (3 rats for saline group, 6 rats for control polymeric vehicle). Additional blood samples are collected once weekly during the 4-week recovery period for the groups treated with the etonogestrel formulation. DMSO serum levels are measured as well and blood samples are collected at predose and then at T0.5 h, T2 h, T3 h, T12 h and T24 h after the first and last administration for the groups treated with the etonogestrel formulation and the polymeric vehicle, and only until T2 h after administration for the control saline-treated group.

All Blood samples are processed by centrifugation for serum collection. Serum samples are then stored at −80° C. until analysis to determine the concentration of etonogestrel and/or DMSO using validated bioanalytical methods. Toxicokinetic evaluation is performed using the measured concentrations. Based on etonogestrel serum concentrations, time and dose effects will be determined by respectively calculating the accumulation ratios for AUCₜₐᵤ, Cₘₐₓ, Cₗₐₛₜ between administrations and the dose proportionality by comparing AUCₜₐᵤ and Cₘₐₓ to corresponding dose ratios.

After 26 or 30 weeks (or more when applicable), animals are euthanized and necropsy procedures, histology processing, and microscopic evaluation are performed.

TABLE 7
Experimental design

| | | DMSO Dose | DMSO | API Dose | API | Dose |
|---|---|---|---|---|---|---|
| Group | | Level | Concentration | Level | Concentration | Volume |
| No | Test item | (mg/kg/adm) | (mg/mL) | (mg/kg/adm) | (mg/mL) | (mL/kg/adm) |
| 1 | Control saline | 0 | 0 | 0 | 0 | 1.380 |
| | (NaCl 0.9%) | | | | | |
| 2 | Control | 960 | 696 | 0 | 0 | 1.380 |
| | vehicle | | | | | |
| 3 | API | 28 | 609 | 4 | 87 | 0.046 |
| | formulation | | | | | |
| 4 | API | 280 | 609 | 40 | 87 | 0.460 |
| | formulation | | | | | |
| 5 | API | 840 | 609 | 120 | 87 | 1.380 |
| | formulation | | | | | |

Having regard to this toxicology study in rats, the low dose level of 4 mg/kg in rats is considered to correspond to a clinically appropriate human dose (e.g., 40 mg/person) based on body surface area. The high dose level of 120 mg/kg represents a 30-fold higher dose, and is considered to produce a sufficient safety margin.

1. An injectable formulation for sustained release of etonogestrel, comprising:
a triblock copolymer (TB) having the formula A-B-A′, wherein:
blocks A and A′ are formed of poly(D,L-lactic acid), and comprise, respectively, m and n lactic acid (LA) repeating units;
block B is formed of poly(ethylene glycol) comprising p ethylene oxide (EO) repeating units; and
the molar ratio R(TB) of LA to EO repeating units ((m+n)/p) is 1.5 to 9;
a diblock copolymer (DB) having the formula C-D, wherein:
block C is formed of methoxypoly(ethylene glycol) comprising r EO repeating units;
block D is formed of poly(D,L-lactic acid) comprising q LA repeating units; and
the molar ratio R(DB) of LA to EO repeating units (q/r) is 1.5 to 15;
a water-miscible, pharmaceutically acceptable organic solvent comprising DMSO; and
etonogestrel, or a prodrug, or salt thereof.

2. The formulation according to claim 1, wherein block B has a number-average molecular weight (Mₙ(B)) of 500 to 3,500 g/mol; and block C has a number-average molecular weight (Mₙ(C)) of 175 to 3,000 g/mol.

3. The formulation according to claim 1, wherein the etonogestrel content is 2.5% w/w or more and 15% w/w or less, relative to the total weight of the formulation.

4. The formulation according to claim 1, wherein the triblock copolymer (TB) content is 8% w/w or more and 40% w/w or less, relative to the total weight of the formulation; and the diblock copolymer (DB) content is 6% w/w or more and 50% w/w or less, relative to the total weight of the formulation.

5. The formulation according to claim 1, wherein the total content of polymers TB and DB in the formulation is 14% w/w or more and 55% w/w or less, relative to the total weight of the formulation.

6. The formulation according to claim 1, wherein the weight ratio of triblock to diblock copolymer (TB:DB) is 10:90 w/w or more and 70:30 w/w or less.

7. The formulation according to claim 1, wherein the water-miscible, pharmaceutically acceptable organic solvent content is 40% w/w or more and 82.5% w/w or less, relative to the total weight of the formulation; and wherein the water-miscible, pharmaceutically acceptable organic solvent consists essentially of DMSO, or is a mixture of DMSO and one or more co-solvents.

8. The formulation according to claim 1, which is in the form of an injectable liquid and/or is capable of passing through a filter with a maximum pore size of 20 μm or less.

9. The formulation according to claim 1, wherein Mₙ(B) is 650 to 3,200 g/mol; R(TB) is 2.5 to 7.5; Mₙ(C) is 200 to 3,000 g/mol; and R(DB) is 1.5 to 11.

10. The formulation according to claim 1, wherein:
(a) Mₙ(B) is 650 to 2,000 g/mol; R(TB) is 2.5 to 5.5; Mₙ(C) is 1,000 to 3,000 g/mol; R(DB) is 1.5 to 4.5; the triblock copolymer (TB) content is 10 to 25% w/w, 15 to 20% w/w, 15 to 18% w/w, or 18 to 20% w/w, relative to the total weight of the formulation; the diblock copolymer (DB) content is 10 to 25% w/w, 15 to 20% w/w, 15 to 18% w/w, or 18 to 20% w/w, relative to the total weight of the formulation; the total content of polymers TB and DB is 20 to 50% w/w, 30 to 40% w/w, or 36 to 40% w/w; and the weight ratio of triblock to diblock copolymer (TB:DB) is 40:60 to 60:40 w/w; or
(b) Mₙ(B) is 650 to 2,000 g/mol; R(TB) 4.5 to 7.5; Mₙ(C) is 200 to 800 g/mol; R(DB) is 6.5 to 11; the triblock copolymer (TB) content is 15 to 25% w/w, relative to the total weight of the formulation; the diblock copolymer (DB) content is 15 to 25% w/w, relative to the total weight of the formulation; the total content of polymers TB and DB is 30 to 50% w/w; and the weight ratio of triblock to diblock copolymer (TB:DB) is 40:60 to 60:40 w/w; or
(c) Mₙ(B) is 1,500 to 3,200 g/mol; R(TB) is 2.5 to 5.5; Mₙ(C) is 1,000 to 3,000 g/mol; R(DB) is 1.5 to 4.5; the triblock copolymer (TB) content is 15 to 25% w/w, relative to the total weight of the formulation; the diblock copolymer (DB) content is 15 to 25% w/w, relative to the total weight of the formulation; the total content of polymers TB and DB is 30 to 50% w/w; and the weight ratio of triblock to diblock copolymer (TB:DB) is 40:60 to 60:40 w/w; or
(d) Mₙ(B) is 650 to 2,000 g/mol; R(TB) 4.5 to 7.5; Mₙ(C) is 200 to 800 g/mol; R(DB) is 6.5 to 11; the triblock copolymer (TB) content is 15 to 25% w/w, relative to the total weight of the formulation; the diblock copolymer (DB) content 10 to 25% w/w, relative to the total weight of the formulation; the total content of polymers TB and DB is 25 to 50% w/w; the weight ratio of triblock to diblock copolymer (TB:DB) is 40:60 to 60:40 w/w; or
(e) Mₙ(B) is 500 to 2,000 g/mol; R(TB) 4.5 to 7.5; Mₙ(C) is 200 to 800 g/mol; R(DB) is 6.5 to 11; the triblock copolymer (TB) content is 10 to 25% w/w, relative to the total weight of the formulation; the diblock copolymer (DB) content is 10 to 25% w/w, relative to the total weight of the formulation; the total content of polymers TB and DB is 20 to 50% w/w; the weight ratio of triblock to diblock copolymer (TB:DB) is 40:60 to 60:40 w/w; or
(f) Mₙ(B) is 500 to 2,000 g/mol; R(TB) 4.5 to 7.5; Mₙ(C) is 200 to 800 g/mol; R(DB) is 6.5 to 11; the triblock copolymer (TB) content is 10 to 25% w/w, relative to the total weight of the formulation; the diblock copolymer (DB) content is 15 to 25% w/w, relative to the total weight of the formulation; the total content of polymers TB and DB is 25 to 50% w/w; the weight ratio of triblock to diblock copolymer (TB:DB) is 40:60 to 60:40 w/w.

11. A pharmaceutical dosage form comprising the formulation according to claim 1.

12. The formulation according to claim 1, which is stable for at least 3 months when stored at 30° C. or at 40° C. with a relative humidity of 75%.

13. A method for preparing the formulation according to claim 1, comprising the steps of:
(i) mixing:
a triblock copolymer (TB) having the formula A-B-A′, wherein blocks A and A′ are formed of poly(D,L-lactic acid), and comprise, respectively, m and n lactic acid (LA) repeating units; block B is formed of poly(ethylene glycol) comprising p ethylene oxide (EO) repeating units; the molar ratio R(TB) of LA to EO repeating units (m+n)/p is 1.5 to 9;
a diblock copolymer (DB) having the formula C-D, wherein block C is formed of methoxypoly(ethylene glycol) comprising r EO repeating units; block D is formed of poly(D,L-lactic acid) comprising q LA repeating units; and the molar ratio R(DB) of LA to EO repeating units (q/r) is 1.5 to 12; and
a water-miscible, pharmaceutically acceptable organic solvent comprising DMSO,
until the polymers are dissolved, thereby obtaining a vehicle; and
(ii) mixing the vehicle with etonogestrel, thereby obtaining the formulation;
(iii) optionally sterilizing the formulation; and
(iv) providing, and optionally packaging, a predetermined amount of the formulation, thereby obtaining the dosage form.

14. An application device comprising the formulation according to claim 1, the device being adapted for administering the formulation or dosage form to a subject by injection.

15. A method for contraception comprising administering an effective amount of the formulation according to claim 1 to a female subject in need thereof.

16. The dosage form according to claim 11, wherein the total amount of etonogestrel per dosage unit is 5 to 60 mg.

17. The dosage form according to claim 11, wherein the volume of the formulation per dosage unit is 0.05 to 1.5 mL

18. The dosage form according to claim 11, which is stable for at least 3 months when stored at 30° C. or at 40° C. with a relative humidity of 75%.

19. The device according to claim 14, the device being:
(a) a self-administration drug-delivery device, a syringe, or a ready-to-use syringe; and/or
(b) adapted for providing an injected volume of 0.05 to 1.5 mL of the formulation or dosage form.

20. The method according to claim 15, wherein the formulation is administered by means of an application device adapted for administering the formulation to a subject by injection.
